(19) United States
(12) Patent Application Publication
(10) Pub. No.: US 2024/0409538 A1
(43) Pub. Date: Dec. 12, 2024

(54) SUBSTITUTED TRIAZOLOHETEROARYL COMPOUNDS AS USP1 INHIBITORS AND THE USE THEREOF

(71) Applicant: IMPACT THERAPEUTICS (SHANGHAI), INC., Shanghai (CN)

(72) Inventors: Sui Xiong CAI, Shanghai (CN); Ye Edward TIAN, Shanghai (CN); Xiaozhu WANG, Jiangsu (CN); Letian ZHANG, Jiangsu (CN)

(21) Appl. No.: 18/702,540

(22) PCT Filed: Oct. 19, 2022
(86) PCT No.: PCT/CN2022/126197

§ 371 Date: Apr. 18, 2024

(30) Foreign Application Priority Data
Oct. 19, 2021 (CN) 202111218092.7

(51) Int. Cl. C07D 471/04; A61K 31/437; A61K 31/4985; A61K 31/5025; A61K 31/506; A61K 31/519; A61K 45/06; A61P 35/00; C07D 487/04

(52) U.S. Cl. CPC C07D 471/04; A61K 31/437; A61K 31/4985; A61K 31/5025; A61K 31/506; A61K 31/519; A61K 45/06; A61P 35/00; C07D 487/04

(57) ABSTRACT

The disclosure provides substituted triazoloheteroaryl compounds as represented in Formula (I) and the use thereof: wherein A₁, A₂, B₁, B₂, B₃, B₄, B₅, D₁, D₂, D₃, D₄, L, Cy₁ and Cy₂ are defined herein. The compounds of Formula I are USP1 inhibitors. Therefore, the compounds of the disclosure may be used to treat diseases associated with USP1 regulation, disorders and conditions, such as cancer.

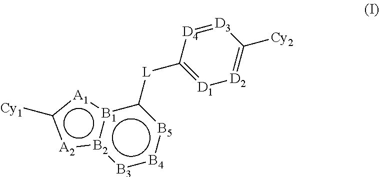

### FIELD OF THE DISCLOSURE

This disclosure is in the field of medicinal chemistry. In particular, the disclosure relates to substituted triazoloheteroaryl compounds, and the use of these compounds as therapeutically effective USP1 inhibitors and anticancer drugs.

### BACKGROUND OF THE INVENTION

Ubiquitin is a 76 amino acid long peptide, which is covalently attached to proteins to modulate their stability, localization, or function. The degradation of a target protein by ubiquitination is a multistep process. The ubiquitination is acted through the sequential action of enzymes such as a ubiquitin activating enzyme (E1), a ubiquitin-conjugating enzyme (E2), and a ubiquitin protein-ligase (E3). Ubiquitination regulates multiple cellular activities as thousands of cellular proteins are ubiquitinated. Ubiquitination is a reversible process. The balance of ubiquitination and deubiquitination is responsible for degree of intracellular protein ubiquitination. Deubiquitinating enzymes (DUBs) greatly contribute to deubiquitination, and DUBs act on ubiquitinated substrates to catalyze the removal of ubiquitin moieties. Thus, the ubiquitination status is a dynamic regulatory mechanism. Ubiquitination also plays a regulatory role in gene expression, cell cycle progression, apoptosis. DNA repair and cell motility, among others (Garcia-Sanstisteban (2013) Mol Cancer 12: 91-103).

Increasing number of studies revealed that protein ubiquitination is emerging as a critical regulatory mechanism underlying DNA damage response (Huang D', Andrea (2006) Mol Cell Biol. 7:323-34). As targeting DDR signaling pathways has become an attractive strategy in oncology, enzymes involved in DNA-damage-induced ubiquitination and deubiquitination could be a potential target for anticancer therapy.

There are 100 genes encoding for deubiquitinases in human. (Garcia-Sanstisteban (2013) Mol Cancer 12: 91-103). One of the best-characterized DUBs is USP1 (ubiquitin-specific protease 1), which encodes a 785 amino acid protein with a predicted molecular weight of 88.2 KDa. USP1 has been identified as a key regulator in the DNA repair processes, mainly in FA (Fanconi Anemia) pathway and Translesion Synthesis (TLS) pathway. USP1 regulates DNA repair through Fanconi anemia (FA)-BRCA pathway by deubiquitylating DNA repair proteins, FANCD2-Ub (Nijman et al. (2005) Mol Cell 17: 331-39). Loss of USP1 function results in an accumulation of FANCD2, which inhibits FA-BRCA-mediated DNA damage repair pathways, leading to elevation of the sensitivity of cancer cells to DNA cross-linking agents, such as mitomycin C and cisplatin. PCNA (Proliferating Cell Nuclear Antigen) is another ubiquitinated substrates of USP1, whose ubiquitination is important for DNA translesion synthesis mechanism (Huang et al. (2006) Nature Cell Biol. 8(4): 339-47). Inhibiting USP1 activity by inhibitor can elevate the sensitivity of cancer cells to DNA cross-linking agents and PARP inhibitors.

USP1 inhibitors can be used in cancer therapy alone or in combination with other DNA damaging agents. The inhibitions of USP1 can impair DNA damage repair pathways. One of the hallmarks of tumor cells is genetic instability, which make tumor cells more sensitive to the DNA damage repairing. Some research reveals that USP1 inhibitor not only can be used as anticancer drugs but also can increase sensitivity to radiotherapy. Further support for advancing USP1 inhibitors shows that USP1 inhibitor also can be used to treat cancer by synthetic lethal mechanism in combination with targeted drugs, such as PARP inhibitors.

Thomas S. et al found ML323 and related N-benzyl-2-phenylpyrimidin-4-amine derivatives displayed excellent inhibitory activity toward USP1/UAF1 by screening (Thomas S. et al. (2014) J. Med. Chem. 57: 8099-8110). The results indicated a strong correlation between compound IC₅₀ values for USP1/UAF1 inhibition and activity in non-small cell lung cancer cells, specifically increased monoubiquitinated PCNA (Ub-PCNA) levels and decreased cell survival. The results established the druggability of the USP1/UAF1 deubiquitinase complex and its potential as a molecular target for anticancer therapies.

Various USP1 inhibitors have been disclosed. For example, WO2014105952, WO2016034675. US20170145012. WO2020139988, WO2020132269. WO2021163530 and WO2022174184A1.

### SUMMARY OF THE DISCLOSURE

The disclosure provides substituted triazoloheteroaryl compounds and analogues as represented in Formula I (including Formula IIa/b and Formula IIIa/b), which can be used as USP1 inhibitors.

The disclosure also provides pharmaceutical compositions comprising an effective amount of the compound of Formula I (including Formula IIa/b and Formula IIIa/b) for the treatment of cancer.

In a specific embodiment, the pharmaceutical composition may also contain one or more pharmaceutically acceptable excipients or carriers or excipients or diluents, for the treatment of cancer.

In a specific embodiment, the pharmaceutical composition may also contain at least one known anticancer drug or pharmaceutically acceptable salts thereof, for the treatment of cancer.

The disclosure is also directed to methods for the preparation of novel compounds of Formula I (including Formula IIa/b and Formula IIIa/b).

### DETAILED DESCRIPTION OF THE DISCLOSURE

It should be understood that the characteristics of the embodiments described herein can be arbitrarily combined to form the technical solution of this disclosure. The definition of each group herein can apply to any of the embodiments described herein. For example, the definitions of the substituents of alkyl herein apply to any of the embodiments described herein unless the substituents of alkyl are clearly defined in the embodiment.

Specifically, the disclosure provides compounds represented by Formula I:

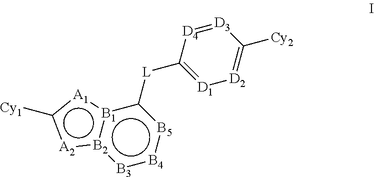

or stereoisomers, tautomers, N-oxides, hydrates, solvates, isotope-substituted derivatives, or pharmaceutically acceptable salts thereof, or mixtures thereof, or prodrugs thereof, wherein:
A₁ and A₂ are each independently selected from a group consisting of N and CR₁;
B₁ and B₂ are each independently selected from a group consisting of N and C, and at most one of B₁ and B₂ is N;
B₃, B₄ and B₅ are each independently selected from a group consisting of N and CR₂;
D₁, D₂, D₃ and D₄ are each independently selected from a group consisting of N and CR₃;
L is selected from a group consisting of NR₆, O, S, SO, SO₂, C═O and an alkylene optionally substituted with R₄ and/or R₅;
Cy₁ is selected from a group consisting of an optionally substituted carbocyclic group, an optionally substituted heterocyclic group, an optionally substituted aryl and an optionally substituted heteroaryl;
Cy₂ is selected from a group consisting of an optionally substituted carbocyclic group, an optionally substituted heterocyclic group, an optionally substituted aryl and an optionally substituted heteroaryl;
R₁, R₂ and R₃ are each independently selected from a group consisting of hydrogen, halogen, cyano, an optionally substituted alkyl, an optionally substituted alkoxy, an optionally substituted carbocyclic group, an optionally substituted alkenyl, an optionally substituted alkynyl, and an optionally substituted amino;
R₄ and R₅ are each independently selected from a group consisting of halogen, cyano, an optionally substituted alkyl, an optionally substituted alkoxy, an optionally substituted carbocyclic group, an optionally substituted alkenyl and an optionally substituted alkynyl; or R₄ and R₅ together with the attached C form a ring;
R₆ is selected from a group consisting of hydrogen and an optionally substituted alkyl.

Preferably, in the definition of the above groups of Formula I, an optionally substituted alkyl, an optionally substituted alkoxy, an optionally substituted alkenyl and an optionally substituted alkynyl each are optionally substituted by 1-5 substituents selected from a group consisting of halogen, hydroxyl, NRₐR_{b}, C₁₋₄ alkoxy, halogenated C₁₋₄ alkoxy, carboxyl and cyano, wherein the said Rₐ and R_{b} are independently H or C₁₋₄ alkyl. More preferably, the said groups can be optionally substituted by 1-5 substituents selected from a group consisting of halogen, hydroxyl and NRₐR_{b}, wherein the said Rₐ and R_{b} are independently H or C₁₋₄ alkyl.

Preferably, in the definition of the above groups of Formula I, an optionally substituted carbocyclic group, an optionally substituted aryl, an optionally substituted heteroaryl and an optionally substituted heterocyclic group each are optionally substituted by 1-5 substituents selected from the group of halogen, hydroxyl, NRₐR_{b}, C₁₋₄ alkyl, halogenated C₁₋₄ alkyl, C₁₋₄ alkyl substituted by hydroxyl, C₁₋₄ alkoxy, halogenated C₁₋₄ alkoxy, carboxyl and cyano, wherein the said Rₐ and R_{b} are independently H or C₁₋₄ alkyl. More preferably, the said optionally substituted carbocyclic group, optionally substituted aryl, optionally substituted heteroaryl and optionally substituted heterocyclic group can be optionally substituted by 1-5 substituents selected from a group consisting of halogen, C₁₋₄ alkyl, C₁₋₄ alkoxy, halogenated C₁₋₄ alkyl, halogenated C₁₋₄ alkoxy, hydroxyl and NRₐR_{b}, wherein the said Rₐ and R_{b} are independently H or C₁₋₄ alkyl. Preferably, the carbocyclic group is C₃₋₈cycloalkyl. Preferably, the aryl is a C₆₋₁₄ aryl, more preferably a phenyl. Preferably, the heteroaryl is a C₅₋₁₀ heteroaryl, more preferably a C₅₋₁₀ heteroaryl containing one to three nitrogen atoms in the ring. Preferably, the heterocyclic group is a C₄₋₁₀ heterocyclic group, preferably containing 1 to 3 heteroatoms selected from a group consisting of O, N and S.

In one or more embodiments of the compound of Formula I, both of A₁ and A₂ are N.

In one or more embodiments of the compound of Formula I, at most one of B₁ and B₂ is N. Preferably, B₁ is N, and B₂ is C; or B₁ is C, and B₂ is N.

In one or more embodiments of the compound of Formula I, B₃, B₄ and B₅ are each independently selected from a group consisting of N and CR₂, wherein R₂ is H, halogen, C₁₋₄ alkyl or C₁₋₄ alkoxy. Preferably, B₃, B₄ and B₅ are each independently N or CH. In some embodiments, all of B₃, B₄ and B₅ are CR₂, wherein R₂ is each independently H, halogen or C₁₋₄ alkyl, preferably, all of B₃, B₄ and B₅ are CH. In some embodiments, B₃ is N, both B₄ and B₅ are CR₂, wherein R₂ is each independently H, halogen or C₁₋₄ alkyl, preferably, each R₂ is H. In some embodiments. B₄ is N, both B₃ and B₅ are CR₂, wherein R₂ is each independently H, halogen or C₁₋₄ alkyl, preferably, each R₂ is H. In some embodiments, B₅ is N, both B₃ and B₄ are CR₂, wherein R₂ is each independently H, halogen or C₁₋₄ alkyl, preferably, each R₂ is H.

In one or more embodiments of the compound of Formula I, the fused heteroaromatic bicyclic ring containing A₁, A₂, B₁, B₂, B₃, B₄ and B₅ is selected from the following groups:

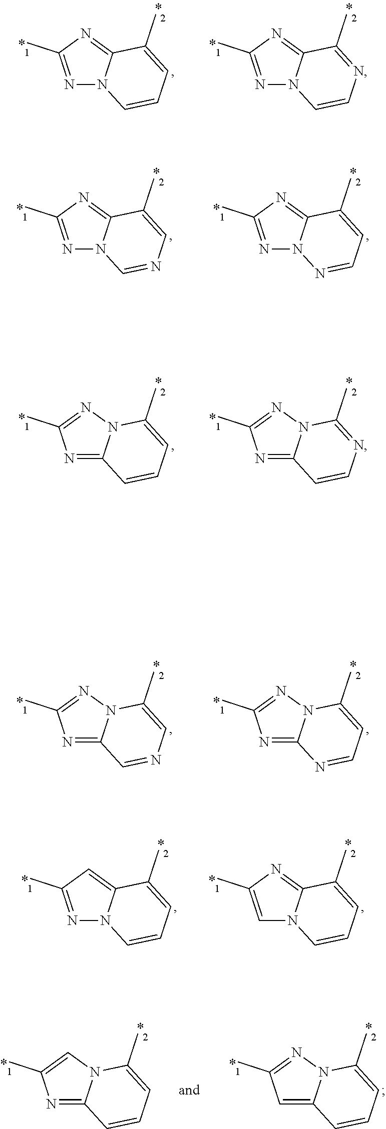

preferably:

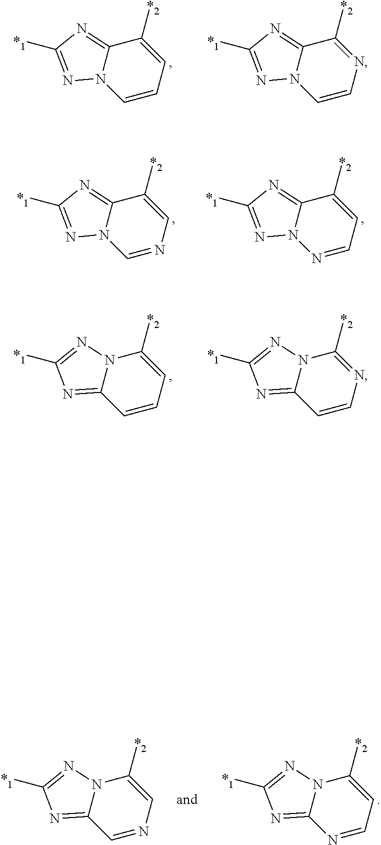

wherein, *1 and *2 refer to the position of attachment of the group to Cy₁ and L of the compound, respectively.

In one or more embodiments of the compound of Formula I, L is an alkylene group, NH, N—C₁₋₃ alkyl or O, preferably a C₁₋₃ alkylene group, more preferably a methylene group or a —CH(CH₃)— group.

In one or more embodiments of the compound of Formula I, D₁, D₂, D₃ and D₄ are CR₃. Preferably, R₃ is selected from a group consisting of hydrogen, halogen, an optionally substituted alkyl and an optionally substituted alkoxy; the said optionally substituted alkyl and optionally substituted alkoxy are preferably an optionally substituted C₁₋₄ alkyl and an optionally substituted C₁₋₄ alkoxy, respectively; preferably, the said alkyl or the said alkoxy is optionally substituted by 1-5 substituents selected from a group consisting of halogen, hydroxyl and NRₐR_{b}, wherein the said Rₐ and R_{b} are independently H or C₁₋₄ alkyl. In some preferred embodiments, D₁ and D₄ are CH. D₂ and D₃ are CR₃, wherein R₃ is each hydrogen, halogen or C₁₋₄ alkoxy; preferably, in some embodiments, at least one of R₃ is a non-hydrogen substituent, i.e., is a halogen or C₁₋₄ alkoxy. In some preferred embodiments. D₁, D₂, D₃ and D₄ are CH. In some embodiments. D₁, D₂, D₃ and D₄ are independently N or CH. In some preferred embodiments, at most 2 of D₁, D₂, D₃ and D₄ are N. In some preferred embodiments, only 2 of D₁, D₂, D₃ and D₄ are N. Preferably, the aryl or heteroaryl containing D₁, D₂, D₃ and D₄ is an optionally substituted phenyl, an optionally substituted pyridyl, an optionally substituted pyrimidinyl or an optionally substituted pyrazinyl; preferably, when the aryl or heteroaryl is substituted, the substituents are selected from the groups described in R₃, including but not limited to halogen, or C₁₋₄ alkyl or C₁₋₄ alkoxyl optionally substituted with 1-5 substituents selected from a group consisting of halogen, hydroxyl and —NRₐR_{b}, wherein the said Rₐ and R_{b} are independently H or C₁₋₄ alkyl.

In one or more embodiments of the compound of Formula I, Cy₁ is an optionally substituted C₃₋₈cycloalkyl, an optionally substituted 4-10 membered heterocyclic group, an optionally substituted 6-14 membered aryl group or an optionally substituted 5-10 membered heteroaryl group. In some further preferred embodiments, the said 5-10 membered heteroaryl group is a nitrogen-containing monocyclic heteroaryl group. Preferably, Cy₁ is an optionally substituted phenyl, an optionally substituted pyridyl, an optionally substituted pyrimidinyl, an optionally substituted pyrazinyl, an optionally substituted pyridazinyl, an optionally substituted piperidinyl, an optionally substituted piperazinyl, an optionally substituted tetrahydrofuranyl, an optionally substituted pyrrolidinyl or an optionally substituted pyrazolyl. Preferably, when Cy₁ is substituted, its substituent(s) are selected from a group consisting of halogen, an optionally substituted C₁₋₄ alkyl, an optionally substituted C₁₋₄ alkoxy, an optionally substituted C₃₋₆ cycloalkyl, an optionally substituted amino and cyano; preferably, each of the said C₁₋₄ alkyl, C₁₋₄ alkoxy and C₃₋₄ cycloalkyl are optionally substituted by 1-5 substituents selected from a group consisting of halogen, hydroxyl and C₁₋₄ alkyl substituted with —NRₐR_{b}, wherein the said Rₐ and R_{b} are independently H or C₁₋₄ alkyl; the said amino is optionally substituted with 1 or 2 C₁₋₄ alkyl.

In one or more embodiments of the compound of Formula I, Cy₂ is an optionally substituted 6-14 membered aryl group, an optionally substituted 5-10 membered heteroaryl group, an optionally substituted C₃₋₈cycloalkyl or an optionally substituted 4-10 membered heterocyclic group. Preferably, Cy₂ is an optionally substituted heteroaryl group, preferably an optionally substituted 5-10 membered nitrogen-containing heteroaryl group, more preferably a 5-membered nitrogen-containing heteroaryl group. In some preferred embodiments, Cy₂ is an optionally substituted imidazolyl or an optionally substituted pyrazolyl. Preferably, when Cy₂ is substituted, its substituent(s) can be 1-5 groups selected from a group consisting of halogen, an optionally substituted C₁₋₄ alkyl, an optionally substituted C₁₋₄ alkoxy and an optionally substituted C₃₋₆ cycloalkyl. Preferably, Cy₂ is imidazolyl substituted with an optionally substituted C₁₋₄ alkyl or pyrazolyl substituted with an optionally substituted C₁₋₄ alkyl. Preferably, each of the said C₁₋₄ alkyl and C₁₋₄ alkoxy are optionally substituted by 1-5 substituents selected from a group consisting of halogen, hydroxyl and C₁₋₄ alkyl substituted with —NRₐR_{b}, wherein the said Rₐ and R_{b} are independently H or C₁₋₄ alkyl. In some further preferred embodiments. Cy₂ is substituted with 1-3 groups selected from a group consisting of C₁₋₄ alkyl, C₃₋₆ cycloalkyl and halogenated C₁₋₄ alkyl. In some further preferred embodiments, Cy₂ is substituted with 1-3 groups selected from a group consisting of C₁₋₄ alkyl and halogenated C₁₋₄ alkyl. In some embodiments. Cy₂ is substituted with a C₁₋₄ alkyl and a halogenated C₁₋₄alkyl. In some embodiments, Cy₂ is imidazolyl substituted with two substituents selected from a group consisting of C₁₋₄ alkyl, C₃₋₆ cycloalkyl and halogenated C₁₋₄ alkyl, with one of its N atom substituted by the C₁₋₄ alkyl.

In one or more embodiments of the compound of Formula I, R₄ and R₅ are each independently selected from a group consisting of halogen and halogenated C₁₋₄ alkyl. In some embodiments, R₄ and R₁ together with the attached C form a 3-5 membered cycloalkyl.

In one or more embodiments of the compound of Formula I, R₆ is H or C₁₋₃ alkyl.

The disclosure provides compounds represented by Formulae IIa and IIb:

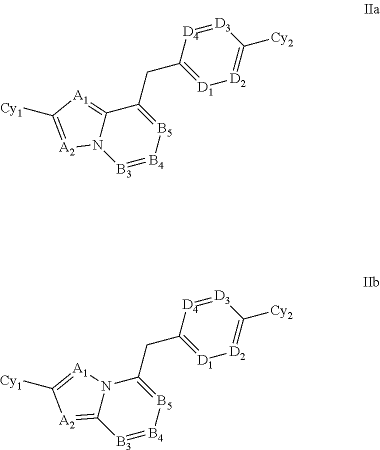

or stereoisomers, tautomers, N-oxides, hydrates, solvates, isotope-substituted derivatives, or pharmaceutically acceptable salts thereof, or mixtures thereof, or prodrugs thereof, wherein:
A₁, A₂, B₃, B₄, B₅, D₁, D₂, D₃, D₄. Cy₁ and Cy₂ are as defined in any embodiments of Formula I.

In one or more embodiments of the compound of Formulae IIa and IIb, both of A₁ and A₂ are N.

In one or more embodiments of the compound of Formulae IIa and IIb, B₃, B₄ and B₅ are each independently selected from a group consisting of N and CR₂; wherein R₂ is H, halogen. C₁₋₄ alkyl or C₁₋₄ alkoxy. Preferably, B₃, B₄ and B₅ are each independently N or CH. In some embodiments, all of B₃, B₄ and B₅ are CR₂, wherein R₂ is each independently H, halogen or C₁₋₄ alkyl, preferably, all of B₃, B₄ and B₅ are CH. In some embodiments, B₃ is N, both B₄ and B₅ are CR₂, wherein R₂ is each independently H, halogen or C₁₋₄alkyl, preferably, each R₂ is H. In some embodiments, B₄ is N, both B₃ and B₅ are CR₂, wherein R₂ is each independently H, halogen or C₁₋₄ alkyl, preferably, each R₂ is H. In some embodiments, B₅ is N, both B₃ and B₄ are CR₂, wherein R₂ is each independently H, halogen or C₁₋₄ alkyl, preferably, each R₂ is H.

In one or more embodiments of the compound of Formulae IIa and IIb, the fused heteroaromatic bicyclic ring containing A₁, A₂, B₃, B₄ and B₅ is selected from the following groups:

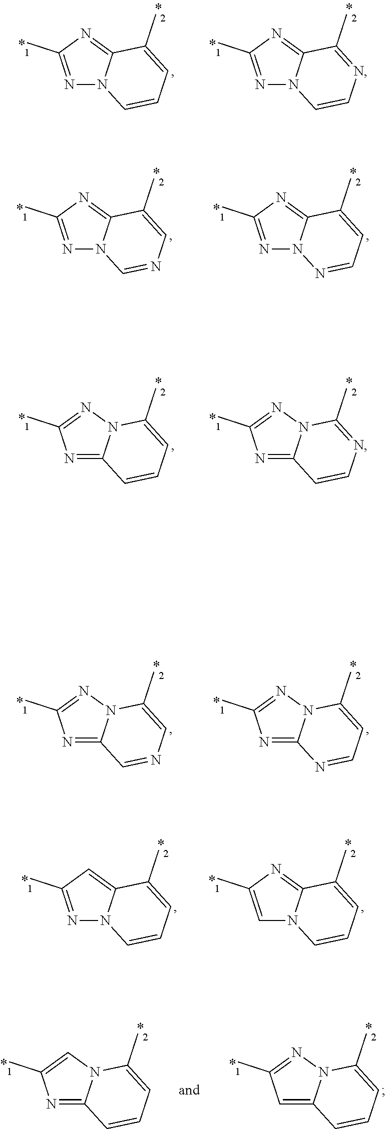

preferably:

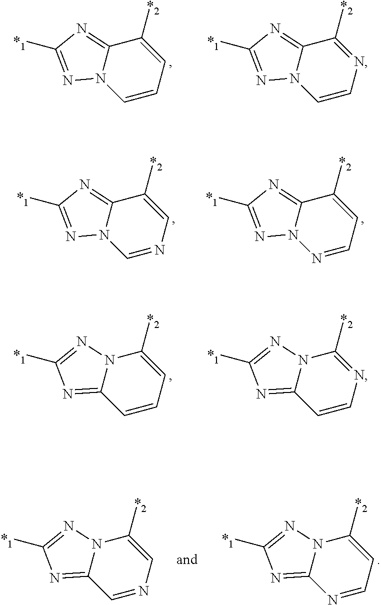

wherein, *1 and *2 refer to the position of attachment of the group to Cy₁ and L of the compound, respectively.

In one or more embodiments of the compound of Formulae IIa and IIb, D₁, D₂, D₃ and D₄ are CR₃. Preferably, R₃ is selected from a group consisting of hydrogen, halogen, an optionally substituted alkyl and an optionally substituted alkoxy; the said optionally substituted alkyl and optionally substituted alkoxy are preferably an optionally substituted C₁₋₄alkyl and an optionally substituted C₁₋₄ alkoxy, respectively; preferably, the said alkyl or the said alkoxy is optionally substituted by 1-5 substituents selected from a group consisting of halogen, hydroxyl and NRₐR_{b}, wherein the said Rₐ and R_{b} are independently H or C₁₋₄ alkyl. In some preferred embodiments, D₁ and D₄ are CH, D₂ and D₃ are CR₃, wherein R₃ is each hydrogen, halogen or C₁₋₄alkoxy; preferably, in some embodiments, at least one of R₃ is a non-hydrogen substituent. i.e., is a halogen or C₁₋₄ alkoxy. In some preferred embodiments. D₁, D₂, D₃ and D₄ are CH. In some embodiments, D₁, D₂, D₃ and D₄ are independently N or CH. In some preferred embodiments, at most 2 of D₁, D₂, D₃ and D₄ are N. In some preferred embodiments, only 2 of D₁, D₂, D₃ and D₄ are N. Preferably, the aryl or heteroaryl containing D₁, D₂, D₃ and D₄ is an optionally substituted phenyl, an optionally substituted pyridyl, an optionally substituted pyrimidinyl or an optionally substituted pyrazinyl; preferably, when the aryl or heteroaryl is substituted, the substituents are selected from the groups described in R₃, including but not limited to halogen, or C₁₋₄ alkyl or C₁₋₄ alkoxyl optionally substituted with 1-5 substituents selected from a group consisting of halogen, hydroxyl and —NRₐR_{b}, wherein the said Rₐ and R_{b} are independently H or C₁₋₄ alkyl.

In one or more embodiments of the compound of Formulae IIa and IIb. Cy₁ is an optionally substituted C₃₋₈cycloalkyl, an optionally substituted 4-10 membered heterocyclic group, an optionally substituted 6-14 membered aryl group or an optionally substituted 5-10 membered heteroaryl group. In some further preferred embodiments, the said 5-10 membered heteroaryl group is a nitrogen-containing monocyclic heteroaryl group. Preferably, Cy₁ is an optionally substituted phenyl, an optionally substituted pyridyl, an optionally substituted pyrimidinyl, an optionally substituted pyrazinyl, an optionally substituted pyridazinyl, an optionally substituted piperidinyl, an optionally substituted piperazinyl, an optionally substituted tetrahydrofuranyl, an optionally substituted pyrrolidinyl or an optionally substituted pyrazolyl. Preferably, when Cy₁ is substituted, its substituent(s) are selected from a group consisting of halogen, an optionally substituted C₁₋₄ alkyl, an optionally substituted C₁₋₄ alkoxy, an optionally substituted C₃₋₆ cycloalkyl, an optionally substituted amino and cyano; preferably, each of the said C₁₋₄ alkyl, C₁₋₄ alkoxy and C₃₋₆ cycloalkyl are optionally substituted by 1-5 substituents selected from a group consisting of halogen, hydroxyl and C₁₋₄ alkyl substituted with —NRₐR_{b}, wherein the said Rₐ and R_{b} are independently H or C₁₋₄ alkyl; the said amino is optionally substituted with 1 or 2 C₁₋₄ alkyl.

In one or more embodiments of the compound of Formulae IIa and IIb, Cy₂ is an optionally substituted 6-14 membered aryl group, an optionally substituted 5-10 membered heteroaryl group, an optionally substituted C₃₋₈ cycloalkyl or an optionally substituted 4-10 membered heterocyclic group. Preferably, Cy₂ is an optionally substituted heteroaryl group, preferably an optionally substituted 5-10 membered nitrogen-containing heteroaryl group, more preferably a 5-membered nitrogen-containing heteroaryl group. In some preferred embodiments, Cy₂ is an optionally substituted imidazolyl or an optionally substituted pyrazolyl. Preferably, when Cy₂ is substituted, its substituent(s) can be 1-5 groups selected from a group consisting of halogen, an optionally substituted C₁₋₄ alkyl, an optionally substituted C₁₋₄ alkoxy and an optionally substituted C₃₋₆ cycloalkyl. Preferably, Cy₂ is imidazolyl substituted with an optionally substituted C₁₋₄ alkyl or pyrazolyl substituted with an optionally substituted C₁₋₄ alkyl. Preferably, each of the said C₁₋₄ alkyl and C₁₋₄ alkoxy are optionally substituted by 1-5 substituents selected from a group consisting of halogen, hydroxyl and C₁₋₄ alkyl substituted with —NRₐR_{b}, wherein the said Rₐ and R_{b} are independently H or C₁₋₄ alkyl. In some further preferred embodiments, Cy₂ is substituted with 1-3 groups selected from a group consisting of C₁₋₄ alkyl, C₃₋₆ cycloalkyl and halogenated C₁₋₄ alkyl. In some further preferred embodiments. Cy₂ is substituted with 1-3 groups selected from a group consisting of C₁₋₄ alkyl and halogenated C₁₋₄ alkyl. In some embodiments. Cy₂ is substituted with a C₁₋₄ alkyl and a halogenated C₁₋₄ alkyl. In some embodiments, Cy₂ is imidazolyl substituted with two substituents selected from a group consisting of C₁₋₄ alkyl, C₃₋₆ cycloalkyl and halogenated C₁₋₄ alkyl, with one of its N atom substituted by the C₁₋₄ alkyl.

In one or more embodiments of the compound of Formulae IIa and IIb. Cy₁ is an optionally substituted phenyl, an optionally substituted pyridyl, an optionally substituted pyrimidinyl, an optionally substituted pyrazinyl, an optionally substituted pyridazinyl or optionally substituted pyrazolyl; preferably. Cy₁ is an optionally substituted phenyl or an optionally substituted pyrimidinyl. Preferably, when Cy₁ is substituted, the number of substituents is 1-5, preferably 1-3, its substituent(s) are selected from a group consisting of halogen, an optionally substituted C₁₋₄ alkyl, an optionally substituted C₁₋₄ alkoxy and C₃₋₆ cycloalkyl; preferably a C₁₋₄ alkyl, C₁₋₄ alkoxy and C₃₋₆ cycloalkyl. The fused heteroaromatic bicyclic ring containing A₁, A₂, B₃, B₄ and B₅ is selected from the following groups:

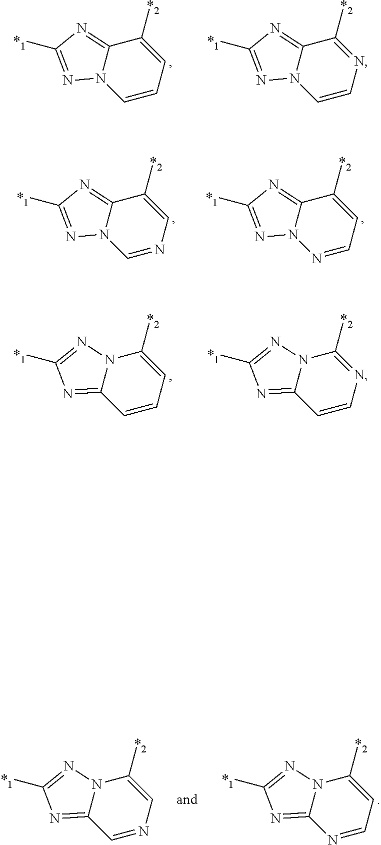

wherein, *1 and *2 refer to the position of attachment of the group to Cy₁ and the methylene group of the compound respectively; the aryl or heteroaryl containing D₁, D₂, D₃ and D₄ is a phenyl optionally substituted by 1-2 substituents selected from a group consisting of halogen. C₁₋₄ alkyl or C₁₋₄ alkoxy; Cy₂ is imidazolyl or pyrazolyl optionally substituted by 1-3 substituents selected from a group consisting of C₁₋₄ alkyl, halogenated C₁₋₄ alkyl and C₃₋₆ cycloalkyl, preferably Cy₂ is imidazolyl substituted with two substituents selected from a group consisting of C₁₋₄ alkyl, C₃₋₆ cycloalkyl and halogenated C₁₋₄ alkyl, with one of its N atom substituted by the C₁₋₄ alkyl.

The disclosure provides compounds represented by Formulae IIIa and IIIb:

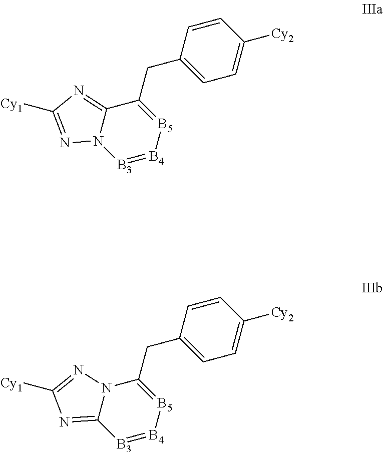

or stereoisomers, tautomers, N-oxides, hydrates, solvates, isotope-substituted derivatives, or pharmaceutically acceptable salts thereof, or mixtures thereof, or prodrugs thereof, wherein:
B₃, B₄, B₅, Cy₁ and Cy₂ are as defined in any embodiments of Formula I or Formula II.

In one or more embodiments of the compound of Formulae IIIa and IIIb, B₃, B₄ and B₅ are each independently selected from a group consisting of N and CR₂; wherein R₂ is H, halogen, C₁₋₄ alkyl or C₁₋₄ alkoxy. Preferably, B₃, B₄ and B₅ are each independently N or CH. In some embodiments, all of B₃, B₄ and B₅ are CR₂, wherein R₂ is each independently H, halogen or C₁₋₄ alkyl, preferably, all of B₃, B₄ and B₅ are CH. In some embodiments. B₃ is N, both B₄ and B₅ are CR₂, wherein R₂ is each independently H, halogen or C₁₋₄ alkyl, preferably, each R₂ is H. In some embodiments, B₄ is N, both B₃ and B₅ are CR₂, wherein R₂ is each independently H, halogen or C₁₋₄ alkyl, preferably, each R₂ is H. In some embodiments, B₅ is N, both B₃ and B₄ are CR₂, wherein R₂ is each independently H, halogen or C₁₋₄ alkyl, preferably, each R₂ is H.

In one or more embodiments of the compound of Formulae IIIa and IIIb, the fused heteroaromatic bicyclic ring containing B₃, B₄ and B₅ is selected from the following groups:

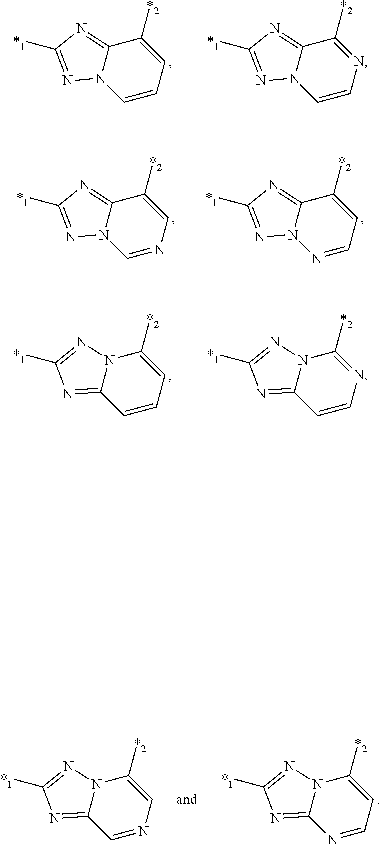

wherein, *1 and *2 refer to the position of attachment of the group to Cy₁ and the rest of the compound, respectively.

In one or more embodiments of the compound of Formulae IIIa and IIIb. Cy₁ is an optionally substituted C₃₋₈cycloalkyl, an optionally substituted 4-10 membered heterocyclic group, an optionally substituted 6-14 membered aryl group or an optionally substituted 5-10 membered heteroaryl group. In some further preferred embodiments, the said 5-10 membered heteroaryl group is a nitrogen-containing monocyclic heteroaryl group. Preferably, Cy₁ is an optionally substituted phenyl, an optionally substituted pyridyl, an optionally substituted pyrimidinyl, an optionally substituted pyrazinyl, an optionally substituted pyridazinyl, an optionally substituted piperidinyl, an optionally substituted piperazinyl, an optionally substituted tetrahydrofuranyl, an optionally substituted pyrrolidinyl or an optionally substituted pyrazolyl. Preferably, when Cy₁ is substituted, its substituent(s) are selected from a group consisting of halogen, an optionally substituted C₁₋₄alkyl, an optionally substituted C₁₋₄ alkoxy, an optionally substituted C₃₋₆ cycloalkyl, an optionally substituted amino and cyano; preferably, each of the said C₁₋₄ alkyl, C₁₋₄ alkoxy and C₃₋₆ cycloalkyl are optionally substituted by 1-5 substituents selected from a group consisting of halogen, hydroxyl and C₁₋₄alkyl substituted with —NRₐR_{b}, wherein the said Rₐ and R_{b} are independently H or C₁₋₄ alkyl; the said amino is optionally substituted with 1 or 2 C₁₋₄ alkyl.

In one or more embodiments of the compound of Formulae IIIa and IIIb. Cy₂ is an optionally substituted 6-14 membered aryl group, an optionally substituted 5-10 membered heteroaryl group, an optionally substituted C₃₋₈ cycloalkyl or an optionally substituted 4-10 membered heterocyclic group. Preferably, Cy₂ is an optionally substituted heteroaryl group, preferably an optionally substituted 5-10 membered nitrogen-containing heteroaryl group, more preferably a 5-membered nitrogen-containing heteroaryl group. In some preferred embodiments, Cy₂ is an optionally substituted imidazolyl or an optionally substituted pyrazolyl. Preferably, when Cy₂ is substituted, its substituent(s) can be 1-5 groups selected from a group consisting of halogen, an optionally substituted C₁₋₄ alkyl, an optionally substituted C₁₋₄ alkoxy and an optionally substituted C₃₋₆ cycloalkyl. Preferably, Cy₂ is imidazolyl substituted with an optionally substituted C₁₋₄ alkyl or pyrazolyl substituted with an optionally substituted C₁₋₄ alkyl. Preferably, each of the said C₁₋₄ alkyl and C₁₋₄ alkoxy are optionally substituted by 1-5 substituents selected from a group consisting of halogen, hydroxyl and C₁₋₄ alkyl substituted with —NRₐR_{b}, wherein the said Rₐ and R_{b} are independently H or C₁₋₄ alkyl. In some further preferred embodiments, Cy₂ is substituted with 1-3 groups selected from a group consisting of C₁₋₄ alkyl, C₃₋₆ cycloalkyl and halogenated C₁₋₄ alkyl. In some further preferred embodiments. Cy₂ is substituted with 1-3 groups selected from a group consisting of C₁₋₄ alkyl and halogenated C₁₋₄ alkyl. In some embodiments, Cy₂ is substituted with a C₁₋₄ alkyl and a halogenated C₁₋₄ alkyl. In some embodiments, Cy₂ is imidazolyl substituted with two substituents selected from a group consisting of C₁₋₄ alkyl, C₃₋₆ cycloalkyl and halogenated C₁₋₄ alkyl, with one of its N atom substituted by the C₁₋₄ alkyl.

In one or more embodiments of the compound of Formulae IIIa and IIIb. Cy₁ is an optionally substituted phenyl, an optionally substituted pyridyl, an optionally substituted pyrimidinyl, an optionally substituted pyrazinyl, an optionally substituted pyridazinyl or optionally substituted pyrazolyl, preferably, Cy₁ is an optionally substituted phenyl or an optionally substituted pyrimidinyl. Preferably, when Cy₁ is substituted, the number of substituents is 1-5, preferably 1-3, its substituent(s) are selected from a group consisting of halogen, an optionally substituted C₁₋₄ alkyl, an optionally substituted C₁₋₄ alkoxy and C₃₋₆ cycloalkyl; preferably a C₁₋₄ alkyl, C₁₋₄ alkoxy and C₃₋₆ cycloalkyl. The fused heteroaromatic bicyclic ring containing B₃, B₄ and B₅ is selected from the following groups:

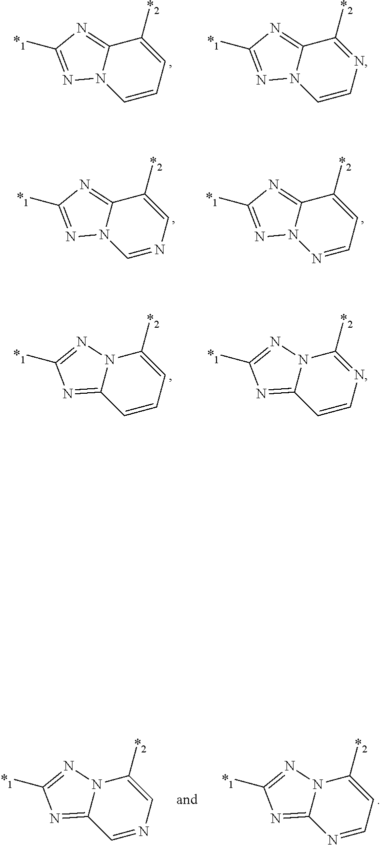

wherein, *1 and *2 refer to the position of attachment of the group to Cy₁ and the methylene of the compound respectively; the aryl or heteroaryl containing D₁, D₂, D₃ and D₄ is a phenyl optionally substituted by 1-2 substituents selected from a group consisting of halogen. C₁₋₄ alkyl or C₁₋₄alkoxy; Cy₂ is imidazolyl or pyrazolyl optionally substituted by 1-3 substituents selected from a group consisting of C₁₋₄ alkyl, halogenated C₁₋₄ alkyl and C₃₋₆ cycloalkyl, preferably Cy₂ is imidazolyl substituted with two substituents selected from a group consisting of C₁₋₄ alkyl, C₃₋₆ cycloalkyl and halogenated C₁₋₄ alkyl, with one of its N atom substituted by the C₁₋₄ alkyl.

In one or more of the foregoing embodiments, preferred compounds of Formula I (including Formulae IIa/b and Formulae IIIa/b) include, without limitation:
8-(4-(1,4-dimethyl-1H-imidazol-2-yl)benzyl)-2-(2-isopropylphenyl)-[1,2,4]triazolo[1,5-a]pyridine (Example 1);
8-(4-(1,4-dimethyl-1H-imidazol-2-yl)benzyl)-2-(2-methoxyphenyl)-[1,2,4]triazolo[1,5-a]pyridine (Example 2);
2-(2-isopropylphenyl)-8-(4-(1-methyl-4-(trifluoromethyl)-1H-imidazol-2-yl)benzyl)-[1,2,4]triazolo[1,5-a]pyridine (Example 3);
2-(4-cyclopropyl-6-methoxypyrimidin-5-yl)-8-(4-(1-isopropyl-4-(trifluoromethyl)-1H-imidazol-2-yl)benzyl)-[1,2,4]triazolo[1,5-a]pyridine (Example 4);
2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-a]pyridine (Example 5);
2-(4-cyclopropyl-6-methoxypyrimidin-5-yl)-8-(4-(5-methyl-3-(trifluoromethyl)-1H-pyrazol-1-yl)benzyl)-[1,2,4]triazolo[1,5-a]pyridine (Example 6);
2-(2-isopropylphenyl)-8-(4-(1-methyl-4-(trifluoromethyl)-1H-imidazol-2-yl)benzyl)-[1,2,4]triazolo[1,5-a]pyrazine (Example 7);
2-(2-isopropylphenyl)-8-(4-(1-methyl-4-(trifluoromethyl)-1H-imidazol-2-yl)benzyl)-[1,2,4]triazolo[1,5-c]pyrimidine (Example 8);
2-(2-isopropylphenyl)-8-(4-(1-methyl-4-(trifluoromethyl)-1H-imidazol-2-yl)benzyl)-[1,2,4]triazolo[1,5-b]pyridazine (Example 9);
2-(2-isopropylphenyl)-5-(4-(1-methyl-4-(trifluoromethyl)-1H-imidazol-2-yl)benzyl)-[1,2,4]triazolo[1,5-a]pyridine (Example 10);
2-(2-isopropylphenyl)-7-(4-(1-methyl-4-(trifluoromethyl)-1H-imidazol-2-yl)benzyl)-[1,2,4]triazolo[1,5-a]pyrimidine (Example 11);
2-(2-isopropylphenyl)-5-(4-(1-methyl-4-(trifluoromethyl)-1H-imidazol-2-yl)benzyl)-[1,2,4]triazolo[1,5-a]pyrazine (Example 12);
2-(2-isopropylphenyl)-5-(4-(1-methyl-4-(trifluoromethyl)-1H-imidazol-2-yl)benzyl)-[1,2,4]triazolo[1,5-c]pyrimidine (Example 13);
2-(4-cyclopropyl-6-methoxypyrimidin-5-yl)-5-(4-(1-methyl-4-(trifluoromethyl)-1H-imidazol-2-yl)benzyl)-[1,2,4]triazolo[1,5-a]pyridine (Example 14);
2-(1-isopropyl-4-methyl-1H-pyrazol-5-yl)-5-(4-(1-methyl-4-(trifluoromethyl)-1H-imidazol-2-yl)benzyl)-[1,2,4]triazolo[1,5-a]pyridine (Example 15);
2-(1-isopropyl-4-methyl-1H-pyrazol-5-yl)-8-(4-(1-methyl-4-(trifluoromethyl)-1H-imidazol-2-yl)benzyl)-[1,2,4]triazolo[1,5-a]pyridine (Example 16);
2-(1-isopropyl-4-methyl-1H-pyrazol-5-yl)-8-(4-(1-methyl-4-(trifluoromethyl)-1H-imidazol-2-yl)benzyl)-[1,2,4]triazolo[1,5-b]pyridazine (Example 17);
2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-a]pyrazine (Example 18);
2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-c]pyrimidine (Example 19);
2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-b]pyridazine (Example 20);
2-(4-cyclopropyl-6-methoxypyrimidin-5-yl)-5-(4-(1-methyl-4-(trifluoromethyl)-1H-imidazol-2-yl)benzyl)-[1,2,4]triazolo[1,5-c]pyrimidine (Example 21);
2-(4-cyclopropyl-6-methoxypyrimidin-5-yl)-5-(4-(1-methyl-4-(trifluoromethyl)-1H-imidazol-2-yl)benzyl)-[1,2,4]triazolo[1,5-a]pyrazine (Example 22);
2-(4-cyclopropyl-6-methoxypyrimidin-5-yl)-7-(4-(1-methyl-4-(trifluoromethyl)-1H-imidazol-2-yl)benzyl)-[1,2,4]triazolo[1,5-a]pyrimidine (Example 23);
8-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-a]pyridine (Example 24);
2-(4-cyclopropyl-6-methoxypyrimidin-5-yl)-8-(3-fluoro-4-(1-methyl-4-(trifluoromethyl)-1H-imidazol-2-yl)benzyl)-[1,2,4]triazolo[1,5-a]pyridine (Example 25);
2-(4-cyclopropyl-6-methoxypyrimidin-5-yl)-8-(4-(1-ethyl-4-(trifluoromethyl)-1H-imidazol-2-yl)-3-fluoro-5-methoxybenzyl)-[1,2,4]triazolo[1,5-a]pyridine (Example 26);
2-(4,6-dimethoxypyrimidin-5-yl)-8-(4-(1-methyl-4-(trifluoromethyl)-1H-imidazol-2-yl)benzyl)-[1,2,4]triazolo[1,5-a]pyridine (Example 27);
2-(4-cyclobutyl-6-methoxypyrimidin-5-yl)-8-(4-(1-methyl-4-(trifluoromethyl)-1H-imidazol-2-yl)benzyl)-[1,2,4]triazolo[1,5-a]pyridine (Example 28);
8-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-a]pyrazine (Example 29);
2-(4-cyclopropyl-6-methoxypyrimidin-5-yl)-8-(3-fluoro-4-(1-methyl-4-(trifluoromethyl)-1H-imidazol-2-yl)benzyl)-[1,2,4]triazolo[1,5-a]pyrazine (Example 30);
2-(4-cyclopropyl-6-methoxypyrimidin-5-yl)-8-(4-(1-ethyl-4-(trifluoromethyl)-1H-imidazol-2-yl)-3,5-difluorobenzyl)-[1,2,4]triazolo[1,5-a]pyrazine (Example 31);
8-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-c]pyrimidine (Example 32);
2-(4-cyclopropyl-6-methoxypyrimidin-5-yl)-8-(3-fluoro-4-(1-methyl-4-(trifluoromethyl)-1H-imidazol-2-yl)benzyl)-[1,2,4]triazolo[1,5-c]pyrimidine (Example 33);
2-(4-cyclopropyl-6-methoxypyrimidin-5-yl)-8-(4-(1-ethyl-4-(trifluoromethyl)-1H-imidazol-2-yl)-3,5-difluorobenzyl)-[1,2,4]triazolo[1,5-c]pyrimidine (Example 34);
2-(4-cyclopropyl-6-cyanopyrimidin-5-yl)-8-(4-(1-methyl-4-(trifluoromethyl)-1H-imidazol-2-yl)benzyl)-[1,2,4]triazolo[1,5-a]pyridine (Example 35);
2-(4-cyclopropyl-6-methoxypyrimidin-5-yl)-8-(1-(4-(1-methyl-4-(trifluoromethyl)-1H-imidazol-2-yl)phenyl)ethyl)-[1,2,4]triazolo[1,5-a]pyridine (Example 36);
5-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-a]pyridine (Example 37);
2-(4-cyclopropyl-6-methoxypyrimidin-5-yl)-5-(3-fluoro-4-(1-methyl-4-(trifluoromethyl)-1H-imidazol-2-yl)benzyl)-[1,2,4]triazolo[1,5-a]pyridine (Example 38);
2-(4-cyclopropyl-6-methoxypyrimidin-5-yl)-5-(4-(1-ethyl-4-(trifluoromethyl)-1H-imidazol-2-yl)-3-fluoro-5-methoxybenzyl)-[1,2,4]triazolo[1,5-a]pyridine (Example 39);
5-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-c]pyrimidine (Example 40);
2-(4-cyclopropyl-6-methoxypyrimidin-5-yl)-5-(3-fluoro-4-(1-methyl-4-(trifluoromethyl)-1H-imidazol-2-yl)benzyl)-[1,2,4]triazolo[1,5-c]pyrimidine (Example 41);
2-(4-cyclopropyl-6-methoxypyrimidin-5-yl)-5-(4-(1-ethyl-4-(trifluoromethyl)-1H-imidazol-2-yl)-3,5-difluorobenzyl)-[1,2,4]triazolo[1,5-c]pyrimidine (Example 42);
5-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-a]pyrazine (Example 43);
2-(4-cyclopropyl-6-methoxypyrimidin-5-yl)-5-(3-fluoro-4-(1-methyl-4-(trifluoromethyl)-1H-imidazol-2-yl)benzyl)-[1,2,4]triazolo[1,5-a]pyrazine (Example 44);
2-(4-cyclopropyl-6-methoxypyrimidin-5-yl)-5-(4-(1-ethyl-4-(trifluoromethyl)-1H-imidazol-2-yl)-3,5-difluorobenzyl)-[1,2,4]triazolo[1,5-a]pyrazine (Example 45);
2-(4-cyclopropyl-6-cyanopyrimidin-5-yl)-5-(4-(1-methyl-4-(trifluoromethyl)-1H-imidazol-2-yl)benzyl)-[1,2,4]triazolo[1,5-a]pyridine (Example 46);
2-(4-cyclopropyl-6-methoxypyrimidin-5-yl)-5-(1-(4-(1-methyl-4-(trifluoromethyl)-1H-imidazol-2-yl)phenyl)ethyl)-[1,2,4]triazolo[1,5-a]pyridine (Example 47);
   or stereoisomers, tautomers. N-oxides, hydrates, isotope-substituted derivatives, solvates or pharmaceutically acceptable salts thereof, or mixtures thereof, or prodrugs thereof.

The term “hydrogen (H)” as employed herein includes its isotopes D and T.

The term “alkyl” as used herein refers to alkyl itself or a straight or branched chain radical of up to ten carbons. Useful alkyl groups include straight-chain, branched C₁₋₁₀ alkyl groups, preferably C₁₋₆ alkyl groups. In some embodiments, alkyl is C₁₋₄ alkyl. In some embodiments, alkyl is C₁₋₃ alkyl. In some embodiments, alkyl is deuterated C₁₋₃ alkyl. Typical C₁₋₁₀ alkyl groups include methyl, ethyl, propyl, isopropyl, butyl, sec-butyl, tert-butyl, pentyl (such as 3-pentyl), hexyl and octyl groups, which may be optionally substituted.

The term “alkenyl” as used herein refers to a straight or branched chain radical of 2-10 carbon atoms, unless the chain length is limited thereto, wherein there is at least one double bond between two of the carbon atoms in the chain; preferably, C₂₋₆ alkenyl. Typical alkenyl groups include ethenyl, 1-propenyl, 2-propenyl, 2-methyl-1-propenyl, 1-butenyl and 2-butenyl.

The term “alkynyl” as used herein refers to a straight or branched chain radical of 2-10 carbon atoms, unless the chain length is limited thereto, wherein there is at least one triple bond between two of the carbon atoms in the chain; preferably. C₂₋₆ alkynyl. Typical alkynyl groups include ethynyl, 1-propynyl, 1-methyl-2-propynyl, 2-propynyl, 1-butynyl and 2-butynyl.

Useful alkoxy groups include oxygen substituted by the above mentioned C₁₋₁₀ alkyl groups, preferred C₁₋₆ alkyl groups or C₁₋₄ alkyl groups. e.g., methoxy, ethoxy, etc. The alkyl in the alkoxy groups may be optionally substituted. Substituents of alkoxy groups include, without limitation, halogen, morpholino, amino (including alkylamino and dialkylamino), and carboxy (including esters thereof).

Useful amino and optionally substituted amino groups include —NH₂, —NHR′ and —NR′R″, wherein —NHR′ and —NR′R″ each are independently hydrogen, an optionally substituted C₁₋₁₀ alkyl (preferably C₁₋₄ alkyl), an optionally substituted cycloalkyl, an optionally substituted aryl or an optionally substituted heteroaryl. In some embodiments. —NHR′ and —NR′R″ together with the N to which they are attached form an optionally substituted 4-7 membered cyclic amino group, which optionally comprises one or more (such as 2, 3) additional heteroatoms selected from O, N and S.

The term “aryl” as used herein by itself or as part of another group refers to monocyclic, bicyclic or tricyclic aromatic groups containing 6 to 14 carbon atoms. Aryl may be substituted by one or more substituents as described herein.

Useful aryl groups include C₆₋₁₄ aryl groups, preferably C₆₋₁₀ aryl groups. Typical C₆₋₁₄ aryl groups include phenyl, naphthyl, phenanthryl, anthracyl, indenyl, azulyl, biphenyl, biphenylene and fluorenyl.

The term “carbocyclic group” as used herein include cycloalkyl and partially saturated carbocyclic groups. Useful cycloalkyl groups are C₃₋₈cycloalkyl. Typical cycloalkyl groups include cyclopropyl, cyclobutyl, cyclopentyl, cyclohexyl and cycloheptyl. Carbocyclic group may be substituted by one or more substituents as described herein.

Useful partially saturated carbocyclic groups include cycloalkenyl groups, such as C₃₋₈cycloalkenyl groups, e.g., cyclopentenyl, cycloheptenyl and cyclooctenyl.

Useful halo or halogen groups include fluoro, chloro, bromo and iodo.

The term “heterocyclic group” as used herein refers to a saturated or partially saturated 3-7 membered monocyclic, or 7-10 membered bicyclic ring system, which consists of carbon atoms and one to four heteroatoms independently selected from C, N, and S, wherein the nitrogen and/or sulfur heteroatoms can be optionally oxidized and the nitrogen can be optionally quaternized, and the term also includes any bicyclic ring system in which any of the above-defined heterocyclic rings is fused to a benzene ring. The heterocycle can be substituted on carbon atom or nitrogen atom if the resulting compound is stable. Heterocyclic group may be substituted by one or more substituents as described herein. The heterocyclic groups mentioned above also include 5-8 membered heterocycloalkyl groups, i.e., heterocyclic groups in which one or more ring C atoms in the cycloalkyl group are replaced by heteroatoms selected from N, O and S.

Useful saturated or partially saturated heterocyclic groups include tetrahydrofuranyl, tetrahydropyranyl, pyranyl, piperidinyl, piperazinyl, oxetanyl, azetidinyl, 1,4-diazepanyl, pyrrolidinyl, imidazolidinyl, imidazolinyl, indoline, isoindolyl, quinuclidinyl, morpholinyl, isochromanyl, chromanyl, pyrazolidine, pyrazolinyl, Tetrahydroisoquinolyl, tetronoyl, oxadiazolyl, oxazolyl and tetramoyl, which may be optionally substituted by one or more substituents as described herein.

The term “heteroaryl” as used herein refers to a group having 5 to 14 ring atoms, preferably 5 to 10 ring atoms, with 6, 10 or 14 π electrons shared in a cyclic array. Ring atoms are carbon atoms and 1-3 heteroatoms selected from oxygen, nitrogen and sulfur. Heteroaryl may be optionally substituted by one or more substituents as described herein.

Useful heteroaryl groups include thienyl (thiophenyl), benzo[d]isothiazol-3-yl, benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, imidazolyl, pyrazolyl, pyridyl (pyridinyl, including without limitation 2-pyridyl, 3-pyridyl, and 4-pyridyl), pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl. β-carbolinyl, phenanthridinyl, acridinyl, perimidinyl, phenanthrolinyl, phenazinyl, isothiazolyl, phenothiazinyl, isoxazolyl, furazanyl, phenoxazinyl, tetrahydrocyclopenta[c]pyrazol-3-yl, benzoisoxazolyl such as 1,2-benzoisoxazol-3-yl, benzimidazolyl, 2-oxindolyl, thiadiazolyl, 2-oxobenzimidazolyl, imidazopyridazinyl, imidazopyridyl, triazolopyridazinyl, pyrazolopyrimidinyl, pyrrolopyrimidinyl, pyrrolopyridyl, pyrrolopyrazinyl or triazolopyrazinyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

In this disclosure, unless otherwise described, when substituted, the alkyl, cycloalkyl, alkoxy, alkenyl, alkynyl, amino, heterocyclic, aryl or heteroaryl as described in any embodiment herein may be substituted by one or more (such as 1, 2, 3, or 4) substituents selected from a group consisting of halogen, amino, cyano, C₁₋₆ alkoxy, C₁₋₆ alkyl, C₆₋₁₀ aryl, C₃₋₈ cycloalkyl, C₂₋₆ chain alkenyl, C₂₋₆ alkynyl, heterocyclic group, heteroaryl, etc. The substituent itself may also be optionally substituted. Preferred substituents include without limitation cyano, halogenated C₁₋₆ alkyl, halo, amino, halogenated C₁₋₆ alkoxy, C₁₋₆ alkyl and C₃₋₈cycloalkyl.

It should be understood that in each embodiment, when the substituent is cyano, cycloalkyl, heterocyclic group, aryl or heteroaryl, the number thereof is usually 1.

Some of the compounds of the present disclosure may exist as stereoisomers including optical isomers. The disclosure includes all stereoisomers and the racemic mixtures of such stereoisomers as well as the individual enantiomers that may be separated according to methods that are well known to those of ordinary skill in the art.

Examples of pharmaceutically acceptable salts include inorganic and organic acid salts, such as hydrochloride, hydrobromide, phosphate, sulphate, citrate, lactate, tartrate, maleate, fumarate, mandelate and oxalate; and inorganic and organic base salts formed with bases, such as sodium hydroxy, tris(hydroxymethyl)aminomethane (TRIS, tromethamine) and N-methyl-glucamine.

Examples of prodrugs of the compounds of the disclosure include the simple esters of carboxylic acid-containing compounds (e.g., those obtained by condensation with a C₁₋₄ alcohol according to methods known in the art); esters of hydroxy containing compounds (e.g., those obtained by condensation with a C₁₋₄ carboxylic acid, C₃₋₆ diacid or anhydride thereof, such as succinic anhydride and fumaric anhydride according to methods known in the art); imines of amino containing compounds (e.g., those obtained by condensation with a C₁₋₄ aldehyde or ketone according to methods known in the art); carbamate of amino containing compounds, such as those described by Leu, et al., (J. Med. Chem. 42:3623-3628 (1999)) and Greenwald, et al. (J. Med. Chem. 42:3657-3667 (1999)); and acetals and ketals of alcohol-containing compounds (e.g., those obtained by condensation with chloromethyl methyl ether or chloromethyl ethyl ether according to methods known in the art).

The compounds of this disclosure may be prepared using methods known to those skilled in the art, or the novel methods of this disclosure. Specifically, the compounds of this disclosure with Formula I (including Formula Ha/b and Formula IIIa/b) can be prepared as illustrated by the exemplary reaction in Scheme 1. Reaction of methyl 2-aminonicotinate and O-(mesitylsulfonyl)hydroxylamine produced 1,2-diamino-3-(methoxycarbonyl)pyridin-1-ium·2,4,6-trimethylbenzenesulfonate. Reaction of 1,2-diamino-3-(methoxycarbonyl)pyridin-1-ium·2,4,6-trimethylbenzenesulfonate and 2-isopropylbenzaldehyde under the catalysis of KOH produced methyl 2-(2-isopropylphenyl)-[1,2,4]triazolo[1,5-a]pyridine-8-carboxylate. Reaction of methyl 2-(2-isopropylphenyl)-[1,2,4]triazolo[1,5-a]pyridine-8-carboxylate and LiAlH₄ produced (2-(2-isopropylphenyl)-[1,2,4]triazolo[1,5-a]pyridin-8-yl)methanol. Reaction of (2-(2-isopropylphenyl)-[1,2,4]triazolo[1,5-a]pyridin-8-yl)methanol and PBr₃ produced 8-(bromomethyl)-2-(2-isopropylphenyl)-[1,2,4]triazolo[1,5-a]pyridine. Suzuki coupling reaction of 8-(bromomethyl)-2-(2-isopropylphenyl)-[1,2,4]triazolo[1,5-a]pyridine and 1,4-dimethyl-2-(4-(4,4,5,5-tetramethyl-1,3,2-dioxaborolan-2-yl)phenyl)-1H-imidazole under the catalysis of Pd(PPh₃)₂Cl₂ produced the target compound 8-(4-(1,4-dimethyl-1H-imidazol-2-yl)benzyl)-2-(2-isopropylphenyl)-[1,2,4]triazolo[1,5-a]pyridine.

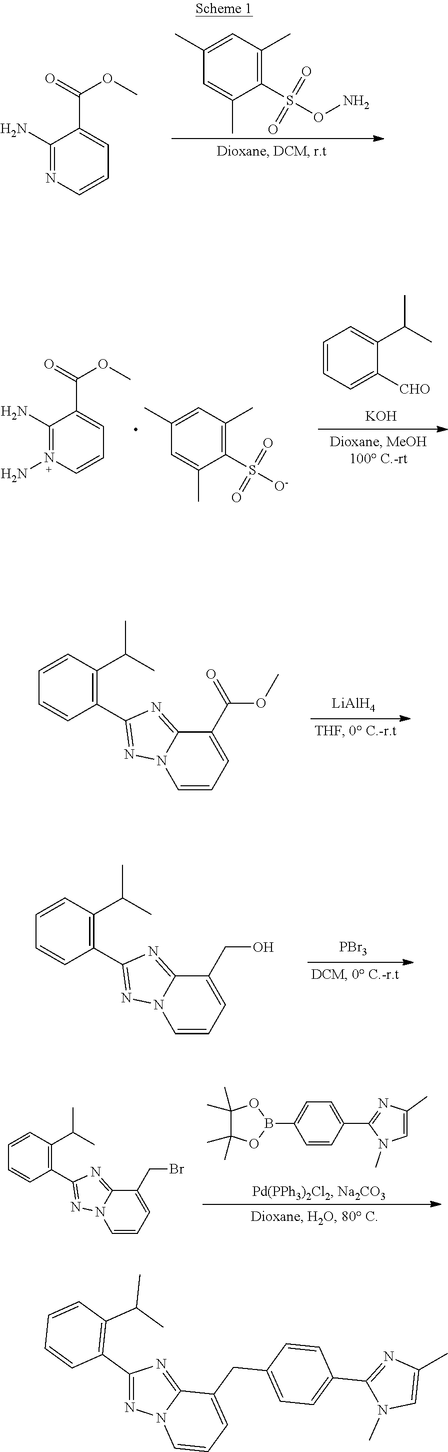

Other related compounds can be prepared using similar methods. For example, replacement of 2-isopropylbenzaldehyde with 2-methoxybenzaldehyde produced the target compound 8-(4-(1,4-dimethyl-1H-imidazol-2-yl)benzyl)-2-(2-methoxyphenyl)-[1,2,4]triazolo[1,5-a]pyridine. Replacement of 1,4-dimethyl-2-(4-(4,4,5,5-tetramethyl-1,3,2-dioxaborolan-2-yl)phenyl)-1H-imidazole with 1-methyl-2-(4-(4,4,5,5-tetramethyl-1,3,2-dioxaborolan-2-yl)phenyl)-4-(trifluoromethyl)-1H-imidazole produced the target compound 2-(2-isopropylphenyl)-8-(4-(1-methyl-4-(trifluoromethyl)-1H-imidazol-2-yl)benzyl)-[1,2,4]triazolo[1,5-a]pyridine.

The compounds of this disclosure can be prepared as illustrated by the exemplary reaction in Scheme 2. Reaction of 6-methylpyridin-2-amine and O-ethyl carbonisothiocyanatidate produced carbamic acid, [[(6-methyl-2-pyridinyl)amino]thioxomethyl]-, ethyl ester. Reaction of carbamic acid, [[(6-methyl-2-pyridinyl)amino]thioxomethyl]-, ethyl ester and hydroxylamine hydrochloride produced 5-methyl-[1,2,4]triazolo[1,5-a]pyridin-2-amine. Sandmeyer reaction of 5-methyl-[1,2,4]triazolo[1,5-a]pyridin-2-amine and NaNO₂/HCl produced 2-chloro-5-methyl-[1,2,4]triazolo[1,5-a]pyridine. Reaction of 2-chloro-5-methyl-[1,2,4]triazolo[1,5-a]pyridine and N-Bromosuccinimide (NBS) under the catalysis of 2,2′-Azobis(2-methylpropionitrile) (AIBN) produced 5-(bromomethyl)-2-chloro-[1,2,4]triazolo[1,5-a]pyridine. Suzuki coupling reaction of 5-(bromomethyl)-2-chloro-[1,2,4]triazolo[1,5-a]pyridine and (4-(1-methyl-4-(trifluoromethyl)-1H-imidazol-2-yl)phenyl)boronic acid under the catalysis of Pd(PPh₃)₂Cl₂ produced 2-chloro-5-(4-(1-methyl-4-(trifluoromethyl)-1H-imidazol-2-yl)benzyl)-[1,2,4]triazolo[1,5-a]pyridine. Suzuki coupling reaction of 2-chloro-5-(4-(1-methyl-4-(trifluoromethyl)-1H-imidazol-2-yl)benzyl)-[1,2,4]triazolo[1,5-a]pyridine and (2-isopropylphenyl)boronic acid under the catalysis of Methanesulfonato(diadamantyl-n-butylphosphino)-2′-amino-1,1′-biphenyl-2-yl)palladium(II) dichloromethane (cataCXium A Pd G3) produced 2-(2-isopropylphenyl)-5-(4-(1-methyl-4-(trifluoromethyl)-1H-imidazol-2-yl)benzyl)-[1,2,4]triazolo[1,5-a]pyridine.

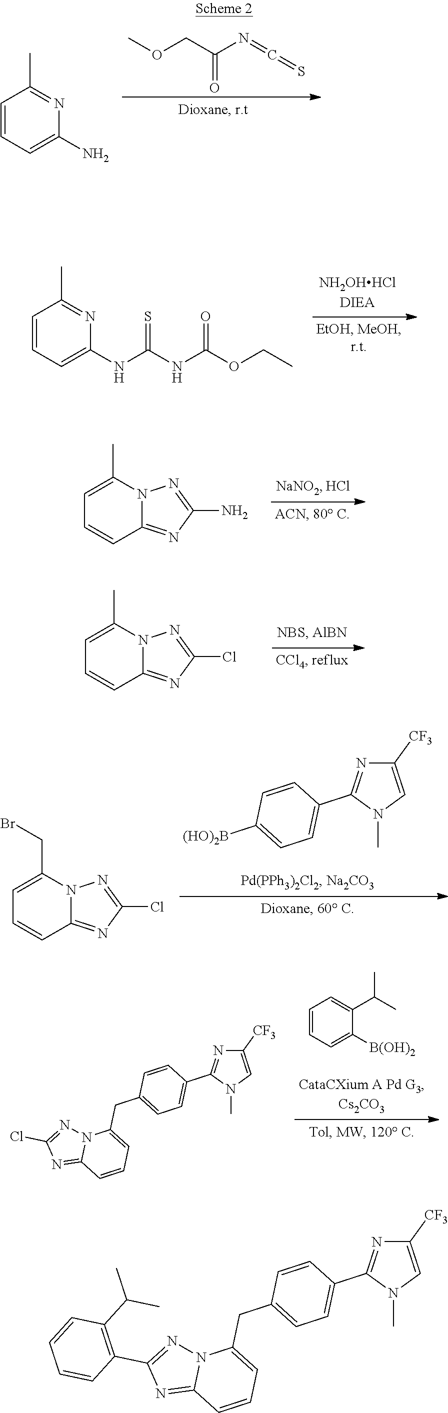

Other related compounds can be prepared using similar methods. For example, replacement of (2-isopropylphenyl)boronic acid with (4-cyclopropyl-6-methoxypyrimidin-5-yl)boronic acid produced the target compound 2-(4-cyclopropyl-6-methoxypyrimidin-5-yl)-5-(4-(1-methyl-4-(trifluoromethyl)-1H-imidazol-2-yl)benzyl)-[1,2,4]triazolo[1,5-a]pyridine. Replacement of (2-isopropylphenyl)boronic acid with (1-isopropyl-4-methyl-1H-pyrazol-5-yl)boronic acid produced the target compound 2-(1-isopropyl-4-methyl-1H-pyrazol-5-yl)-5-(4-(1-methyl-4-(trifluoromethyl)-1H-imidazol-2-yl)benzyl)-[1,2,4]triazolo[1,5-a]pyridine.

The compounds of this disclosure can be prepared as illustrated by the exemplary reaction in Scheme 3. The Negishi cross-coupling reaction of 3-bromopyrazin-2-amine and 2-(4-(bromomethyl)phenyl)-1-methyl-4-(trifluoromethyl)-1H-imidazole under the catalysis of Pd(PPh₃)₄ produced 3-(4-(1-methyl-4-(trifluoromethyl)-1H-imidazol-2-yl)benzyl)pyrazin-2-amine. Reaction of 3-(4-(1-methyl-4-(trifluoromethyl)-1H-imidazol-2-yl)benzyl)pyrazin-2-amine and O-(mesitylsulfonyl)hydroxylamine produced 1,2-diamino-3-(4-(1-methyl-4-(trifluoromethyl)-1H-imidazol-2-yl)benzyl)pyrazin-1-ium·2,4,6-trimethylbenzenesulfonate. Reaction of 1,2-diamino-3-(4-(1-methyl-4-(trifluoromethyl)-1H-imidazol-2-yl)benzyl)pyrazin-1-ium·2,4,6-trimethylbenzenesulfonate and 4-cyclopropyl-6-methoxypyrimidine-5-carbaldehyde under the catalysis of Cs₂CO₃ produced the target compound 2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-a]pyrazine.

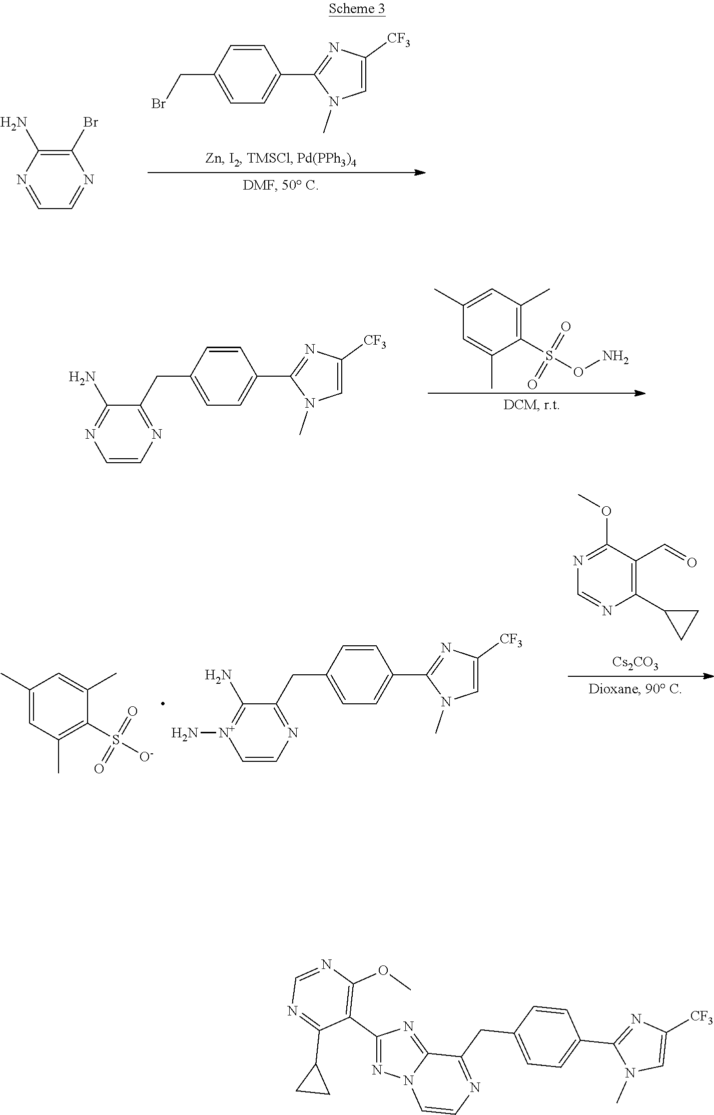

Other related compounds can be prepared using similar methods. For example, replacement of 3-bromopyrazin-2-amine with 5-bromopyrimidin-4-amine produced the target compound 2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-c]pyrimidine. Replacement of 3-bromopyrazin-2-amine with 2-bromopyrimidin-4-amine produced the target compound 2-(4-cyclopropyl-6-methoxypyrimidin-5-yl)-5-(4-(1-methyl-4-(trifluoromethyl)-1H-imidazol-2-yl)benzyl)-[1,2,4]triazolo[1,5-c]pyrimidine. Replacement of 3-bromopyrazin-2-amine with 6-bromopyrazin-2-amine produced the target compound 2-(4-cyclopropyl-6-methoxypyrimidin-5-yl)-5-(4-(1-methyl-4-(trifluoromethyl)-1H-imidazol-2-yl)benzyl)-[1,2,4]triazolo[1,5-a]pyrazine. Replacement of 3-bromopyrazin-2-amine with 4-bromopyrimidin-2-amine produced the target compound 2-(4-cyclopropyl-6-methoxypyrimidin-5-yl)-7-(4-(1-methyl-4-(trifluoromethyl)-1H-imidazol-2-yl)benzyl)-[1,2,4]triazolo[1,5-a]pyrimidine.

One important aspect of the present disclosure is the finding that the compounds of Formula I (including the compounds of Formulae IIa/b and Formulae IIIa/b as described herein) are USP1 inhibitors. Therefore, the compounds of Formula I (including the compounds of Formulae IIa/b and Formulae IIIa/b as described herein) can be used to treat or prevent diseases associated with USP1 regulation, such as cancer; or be used to prepare medicaments for the treatment or prevention of diseases associated with USP1 regulation, such as cancer. The disease related with USP1-adjusted or USP1-mediated refers to the disease in which USP1 is involved in the occurrence and progression of diseases and benefits from diseases in which USP1 activity is inhibited.

The present disclosure also includes methods for the treatment or prevention of diseases associated with USP1 regulation, especially, methods of the treatment or prevention of diseases associated with USP1 regulation and methods of treatment or prevention of diseases caused by defects in DDR function, comprising administering to an object (especially mammal, more specifically human) in need an effective amount of the compound of Formula I (including the compound of Formulae IIa/b and Formulae IIIa/b as described herein) or stereoisomers, tautomers, N-oxides, hydrates, isotope-substituted derivatives, solvates or pharmaceutically acceptable salts thereof, or mixtures thereof, or prodrugs thereof, or a pharmaceutical composition comprising an effective amount of the compound of Formula I (including the compound of Formulae IIa/b and Formulae IIIa/b as described herein) or stereoisomers, tautomers. N-oxides, hydrates, isotope-substituted derivatives, solvates or pharmaceutically acceptable salts thereof, or mixtures thereof, or prodrugs thereof.

In the disclosure, the diseases associated with USP1 regulation include cancers. Preferably, the cancers associated with USP1 regulation have defects in DDR function. The diseases associated with USP1 regulation that can be treated or prevented by the methods or pharmaceutical compositions of the disclosure include without limitation liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, Wilms tumor, cervical cancer, testicular cancer, soft tissue sarcoma, primary macroglobulinemia, bladder cancer, chronic myeloid leukemia, primary brain cancer, malignant melanoma, non-small lung cancer, small cell lung cancer, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostate cancer.

The present disclosure also includes the method for the treatment or prevention of other diseases caused by excessive or abnormal cell proliferation, including proliferative or hyperproliferative diseases, such as myeloproliferative diseases, especially proliferative or hyperproliferative diseases caused by excessive or abnormal cell proliferation related with USP1 regulation. Therefore, the disclosure also includes the compound of Formula I (including the compound of Formulae IIa/b and Formulae IIIa/b as described herein) for the treatment or prevention of other diseases caused by excessive or abnormal cell proliferation, especially proliferative or hyperproliferative diseases caused by excessive or abnormal cell proliferation related with USP1 regulation.

In practicing the therapeutic methods, effective amounts of pharmaceutical preparations are administered to an individual exhibiting the symptoms of one or more of these disorders. The pharmaceutic preparations comprise therapeutically effective concentrations of the compounds of Formula I, Formulae IIa/b or Formulae IIIa/b, formulated for oral, intravenous, local or topical application, for the treatment of cancer and other diseases. The amounts are effective to ameliorate or eliminate one or more symptoms of the disorders. An effective amount of a compound for treating a particular disease is an amount that is sufficient to ameliorate or in some manner reduce the symptoms associated with the disease. Such amount may be administered as a single dosage or may be administered according to an effective regimen. The amount may cure the disease but, typically, is administered in order to ameliorate the symptoms of the disease. Typically, repeated administration is required to achieve the desired amelioration of symptom.

In another embodiment, there is provided a pharmaceutical composition comprising a compound of Formula I. Formulae IIa/b or Formulae IIIa/b as an USP1 inhibitor, or stereoisomers, tautomers. N-oxides, hydrates, solvates, isotope-substituted derivatives, or pharmaceutically acceptable salts thereof and one or more pharmaceutically acceptable excipients or carriers.

Another embodiment of the present disclosure is directed to a pharmaceutical composition effective to treat cancer comprising a compound of Formula I. Formulae IIa/b or Formulae IIIa/b as an USP1 inhibitor, or stereoisomers, tautomers. N-oxides, hydrates, solvates, isotope-substituted derivatives, or pharmaceutically acceptable salts thereof or prodrugs thereof, in combination with at least one known anticancer agent or a pharmaceutically acceptable salt thereof. In particular, the compound herein can be combined with other anticancer drugs related to the mechanism of DNA damage and repair, including PARP inhibitors, such as olaparib, niraprib, rucaparib, talazoparib, pamiparib, fluzoparib and senaparib; HDAC inhibitors such as Volinota, Romididesin, Papiseta and Bailesta; and so on. And the compound herein can be combined with other anticancer drugs related to cell division detection sites, including Chk1/2 inhibitors, CDK4/6 inhibitors such as paposinib, ATM inhibitors, Weel inhibitors, ATR inhibitors, Myt1 inhibitors, DNA-PK inhibitors, and so on. And combination with other targeted anti-cancer drugs, including PRMT5 inhibitors. Polθ inhibitors, RAD51 inhibitors, and so on. Other known anticancer agents which may be used for anticancer combination therapy include, but are not limited to alkylating agents, such as busulfan, melphalan, chlorambucil, cyclophosphamide, ifosfamide, temozolomide, bendamustine, cis-platin, mitomycin C, bleomycin and carboplatin; topoisomerase I inhibitors, such as camptothecin, irinotecan and topotecan; topoisomerase II inhibitors, such as doxorubicin, epirubicin, aclacinomycin, mitoxantrone, elliptinium and etoposide: RNA/DNA antimetabolites, such as 5-azacytidine, gemcitabine, 5-fluorouracil, capecitabine and methotrexate; DNA antimetabolites, such as 5-fluoro-2′-deoxy-uridine, fludarabine, nelarabine, ara-C, pralatrexate, pemetrexed, hydroxyurea and thioguanine; antimitotic agent such as colchicine, vinblastine, vincristine, vinorelbine, paclitaxel, ixabepilone, cabazitaxel and docetaxel; antibodies such as mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, Ofatumumab, Dinutuximab, Blinatumomab, ipilimumab, avastin, herceptin and mabthera; Antibody-Drug Conjugates (ADC) such as T-DM1, Trastuzumab Deruxtecan. Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, Belantamab Mafodotin; kinase inhibitors such as imatinib, gefitinib, erlotinib, osimertinib, afatinib, ceritinib, alectinib, crizotinib, erlotinib, lapatinib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, Idelalisib, pazopanib. Torisel and everolimus. Other known anticancer agents which may be used for anticancer combination therapy include tamoxifen, letrozole, fulvestrant, mitoguazone, octreotide, retinoic acid, arsenic, zoledronic acid, bortezomib, carfilzomib. Ixazomib, vismodegib, sonidegib, denosumab, thalidomide, lenalidomide, Venetoclax, Aldesleukin (recombinant human interleukin-2) and Sipueucel-T (prostate cancer treatment vaccine).

In practicing the methods of the present disclosure, the compound of the disclosure may be administered together with at least one known anticancer agent in a unitary pharmaceutical composition. Alternatively, the compound of the disclosure may be administered separately from at least one known anticancer agent. In one embodiment, the compound of the disclosure and at least one known anticancer agent are administered substantially simultaneously, i.e. all agents are administered at the same time or one after another, provided that compounds reach therapeutic levels in the blood at the same time. In another embodiment, the compound of the disclosure and at least one known anticancer agent are administered according to individual dose schedule, provided that the compounds reach therapeutic levels in the blood.

Another embodiment of the present disclosure is directed to a bioconjugate, which functions as a USP1 inhibitor, that comprises a compound described herein and is effective to inhibit tumor. The bioconjugate that inhibits tumor is consisted of the compound described herein and at least one known therapeutically useful antibody, such as trastuzumab or rituximab, or growth factor, such as EGF or FGF, or cytokine, such as IL-2 or IL-4, or any molecule that can bind to cell surface. The antibodies and other molecules could deliver the compound described herein to its targets, making it an effective anticancer agent. The bioconjugates could also enhance the anticancer effect of the therapeutically useful antibodies, such as trastuzumab or rituximab.

Another embodiment of the present disclosure is directed to a pharmaceutical composition effective to inhibit tumor comprising the USP1 inhibitor of Formula I (including Formulae IIa/b and Formulae IIIa/b), or stereoisomers, tautomers, N-oxides, hydrates, solvates, isotope-substituted derivatives, or pharmaceutically acceptable salts thereof or prodrugs thereof, in combination with radiation therapy. In this embodiment, the compound of the disclosure may be administered at the same time as the radiation therapy or at a different time.

Yet another embodiment of the present disclosure is directed to a pharmaceutical composition effective for post-surgical treatment of cancer, comprising the USP1 inhibitor of Formula I. Formulae IIa/b or Formulae IIIa/b, or stereoisomers, tautomers, N-oxides, hydrates, solvates, isotope-substituted derivatives, or pharmaceutically acceptable salts thereof or prodrugs thereof. The disclosure also relates to a method of treating cancer by surgically removing tumor and then treating the mammal with the pharmaceutical composition described herein.

Pharmaceutical compositions of this disclosure include all pharmaceutical preparations which contain the compounds of the present disclosure in an amount that is effective to achieve its intended purpose. While individual needs vary, determination of optimal amounts of each component in the pharmaceutical preparations is within the skill of the art. Typically, the compounds or the pharmaceutically acceptable salt thereof may be administered to mammals, orally at a dose of about 0.0025 to 50 mg per kg body weight per day. Preferably, from approximately 0.01 mg/kg to approximately 10 mg/kg body weight is orally administered. If a known anticancer agent is also administered, it is administered in an amount that is effective to achieve its intended purpose. The optimal amounts of such known anticancer agents are well known to those skilled in the art.

The unit oral dose may comprise from approximately 0.01 to approximately 50 mg, preferably approximately 0.1 to approximately 10 mg of the compound of the disclosure. The unit dose may be administered one or more times, with one or more tablets daily, each containing from approximately 0.1 to approximately 50 mg, conveniently approximately 0.25 to 10 mg of the compound of the disclosure or its solvates.

In a topical formulation, the compound of the disclosure may be present at a concentration of approximately 0.01 to 100 mg per gram of carrier.

The compound of the disclosure may be administered as a raw chemical. The compounds of the disclosure may also be administered as part of a suitable pharmaceutical preparation containing pharmaceutically acceptable carriers (comprising excipients and auxiliaries), which facilitate the processing of the compounds into pharmaceutically acceptable preparations. Preferably, the pharmaceutical preparations, particularly oral preparations and those used for the preferred administration, such as tablets, dragees, and capsules, as well as solutions suitable for injection or oral administration, contain from approximately 0.01% to 99%, preferably from approximately 0.25% to 75% of active compound(s), together with excipient(s).

Also included within the scope of the present disclosure are the non-toxic pharmaceutically acceptable salts of the compounds of the present disclosure. Acid addition salts are formed by mixing a solution of the compounds of the present disclosure with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Base addition salts are formed by mixing a solution of the compounds of the present disclosure with a solution of a pharmaceutically acceptable non-toxic base, such as sodium hydroxide, potassium hydroxide, choline hydroxide, sodium carbonate, tris(hydroxymethyl)aminomethane. N-methyl-glucamine and the like.

The pharmaceutical preparations of the disclosure may be administered to any mammal, so long as they may experience the therapeutic effects of the compounds of the disclosure. Foremost among such mammals are humans and veterinary animals, although the disclosure is not intended to be so limited.

The pharmaceutical preparations of the present disclosure may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively or concurrently, administration may be by oral route. The dosage administered will be dependent upon the age, health, and weight of the recipient, type of concurrent treatment, frequency of treatment, and the nature of the effect desired.

The pharmaceutical preparations of the present disclosure are manufactured in a known manner. e.g., by means of conventional mixing, granulating, dragee-making, dissolving, or lyophilizing processes. Pharmaceutical preparations for oral use may be obtained by combining the active compounds with solid excipients, optionally grinding the resulting mixture, processing the mixture of granules after adding suitable auxiliaries if desired or necessary, thereby obtaining tablets or dragee cores.

Suitable excipients are, in particular, fillers, such as saccharides. e.g. lactose or sucrose, mannitol or sorbitol; cellulose preparations and/or calcium phosphates, e.g. tricalcium phosphate or calcium hydrogen phosphate; as well as binders, such as starch paste, including. e.g., maize starch, wheat starch, rice starch, potato starch, gelatin, tragacanth, methylcellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, in particular, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropylmethylcellulose phthalate, are used. Dyes or pigments may be added to the tablets or dragee coatings, e.g., for identification or in order to characterize combinations of active compound doses.

Other pharmaceutical preparations, which may be used orally, include push-fit capsules made of gelatin, as well as soft, sealed capsules made of gelatin and a plasticizer, such as glycerol or sorbitol. The push-fit capsules may contain the active compounds in the form of granules, which may be mixed with fillers, such as lactose; binders, such as starches; and/or lubricants, such as talc or magnesium stearate and stabilizers. In soft capsules, the active compounds are preferably dissolved or suspended in suitable liquids, such as fatty oils, or liquid paraffin. In addition, stabilizers may be added.

Suitable formulations for parenteral administration include aqueous solutions of the active compounds. e.g., aqueous solutions and alkaline solutions of water-soluble salts. In addition, suspensions of the active compounds as appropriate oily injection suspensions may be administered. Suitable lipophilic solvents or vehicles include fatty oils. e.g., sesame oil, or synthetic fatty acid esters. e.g., ethyl oleate or triglycerides or polyethylene glycol-400, or cremophor, or cyclodextrins. Aqueous injection suspensions may contain substances which increase the viscosity of the suspension, e.g., sodium carboxymethyl cellulose, sorbitol, and/or dextran. Optionally, suspension stabilizers may also be contained.

In accordance with one aspect of the present disclosure, compounds of the disclosure are employed in topical and parenteral formulations and are used for the treatment of skin cancer.

The topical formulations of this disclosure are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The present disclosure also involves use of the compounds of the disclosure for the preparation of a medicament for the treatment or prevention of clinical symptoms in response to the effect of inhibiting the activity of USP1. The medicament may include the above-mentioned pharmaceutical compositions.

The following examples are illustrative, but not limiting, of the method and compositions of the present disclosure. Other suitable modifications and adaptations of the variety of conditions and parameters normally encountered in clinical therapy and which are obvious to those skilled in the art are within the spirit and scope of the disclosure.

### EXAMPLES

### General Remarks

All reagents were of commercial quality. Solvents were dried and purified by standard methods. Mass spectrum analyses were recorded on a Platform II (Agilent 6110) quadrupole mass spectrometer fitted with an electrospray interface. ¹H NMR spectra was recorded at 400 MHz, on a Brücker Ascend 400 apparatus. Chemical shifts were recorded in parts per million (ppm) downfield from TMS (0.00 ppm), and J coupling constants were reported in hertz (Hz).

### Example 1

### 8-(4-(1,4-dimethyl-1H-imidazol-2-yl)benzyl)-2-(2-isopropylphenyl)-[1,2,4]triazolo[1,5-a]pyridine

a) Preparation of methyl 2-(2-isopropylphenyl)-[1,2,4]triazolo[1,5-a]pyridine-8-carboxylate: To a stirred solution of methyl 2-aminonicotinate (0.6 g, 3.9 mmol) in dioxane (50 mL) was added O-(mesitylsulfonyl)hydroxylamine (1.0 g in 10 mL DCM, 4.7 mmol) under N₂, the mixture was stirred at room temperature for 2 h. Then 2-isopropylbenzaldehyde (0.7 g, 4.7 mmol) was added, and the mixture was stirred at 100° C. for 2 h. The reaction was cooled to room temperature, then KOH (10 mL 1N in MeOH) was added and stirred at room temperature for 1 h. To the mixture was added water and the mixture was extracted with ethyl acetate (30 mL×3). The combined organic layers were washed successively with water twice and saturated brine, dried over anhydrous sodium sulfate, and then filtered and the filtrate was concentrated under reduced pressure to give the title compound (600 mg, white solid, yield 51.5%) used in the next step without further purification. MS (ESI, m/z): 296.1 [M+H]⁺.

b) Preparation of (2-(2-isopropylphenyl)-[1,2,4]triazolo[1,5-a]pyridin-8-yl)methanol: To a solution of methyl 2-(2-isopropylphenyl)-[1,2,4]triazolo[1,5-a]pyridine-8-carboxylate (600.0 mg, 2.0 mmol) in dry THF (60 mL) at 0° C. was added LiAlH₄ (1.6 mL, 4.0 mmol, 2.5 M in toluene) over 10 min. The reaction mixture was warmed to room temperature. After the reaction was completed, the reaction was quenched with water (20 mL), and extracted with ethyl acetate (20 mL×3). The combined organic layers were dried with anhydrous Na₂SO₄, filtered and concentrated to give the title compound (500 mg, white solid, yield 92.1%) used in the next step without further purification. MS (ESI, m/z): 268.1 [M+H]⁺.

c) Preparation of 8-(bromomethyl)-2-(2-isopropylphenyl)-[1,2,4]triazolo[1,5-a]pyridine: To a solution of (2-(2-isopropylphenyl)-[1,2,4]triazolo[1,5-a]pyridin-8-yl)methanol (400 mg, 1.5 mmol) in dry DCM (40 mL) at 0° C. was added PBr₃ (485 mg, 1.7 mmol), and the mixture was stirred at room temperature for 2 h. Then the reaction was quenched with a saturated NaHCO₃ solution (20 mL) and extracted with DCM (20 mL×3). The combined organic layers were dried over anhydrous sodium sulfate, filtered and the filtrate was concentrated under reduced pressure. The crude product was purified by silica gel chromatography (petroleum ether/ethyl acetate=10:1) to give the title compound (270 mg, white solid, yield 54.6%). MS (ESI, m/z): 330.0 [M+H]⁺. ¹H NMR (300 MHz, DMSO-d₆): δ 8.99 (dd, J=6.8, 1.1 Hz, 1H), 7.91-7.79 (m, 2H), 7.55-7.40 (m, 2H), 7.38-7.26 (m, 1H), 7.23-7.18 (m, 1H), 5.01 (s, 2H), 3.97-3.82 (m, 1H), 1.22 (d, J=6.9 Hz, 6H).

d) Preparation of 8-(4-(1,4-dimethyl-1H-imidazol-2-yl)benzyl)-2-(2-isopropylphenyl)-[1,2,4]triazolo[1,5-a]pyridine: A solution of 8-(bromomethyl)-2-(2-isopropylphenyl)-[1,2,4]triazolo[1,5-a]pyridine (200 mg, 0.6 mmol), Na₂CO₃ (193 mg, 1.8 mmol), 1,4-dimethyl-2-(4-(4,4,5,5-tetramethyl-1,3,2-dioxaborolan-2-yl)phenyl)-1H-imidazole (181 mg, 0.6 mmol) and Pd(PPh₃)₂Cl₂ (43 mg, 0.06 mmol) in 1,4-dioxane (20 mL) and water (7 mL) was stirred at 60° C. for 2 h under N₂. The mixture was diluted with water (30 mL) and extracted with EtOAc (30 mL×3), the organic layers were dried over Na₂SO₄, filtered and concentrated, and then purified by prep-HPLC (ACN/water=25%/75%, 0.1% FA as additive) to give the target compound (30 mg, white solid, 11.7%).

The following compounds of Examples 2-9 were prepared using a synthesis method similar to that described in Example 1.

| | | | LC-MS | |
|---|---|---|---|---|
| Example | Compound | MW | (ESI) | ¹H NMR (400 MHz) |
| 1 | 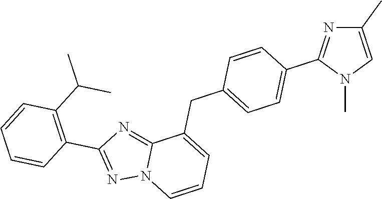 [SMILES: Cc1cn(C)c(-c2ccc(Cc3cccn4nc(-c5ccccc5C(C)C)nc34)cc2)n1] | 421.55 | 422.2 [M + H]⁺ | DMSO-d₆: δ 8.87 (d, J = 6.0 Hz, 1H), 7.82 (dd, J = 7.7, 1.1 Hz, 1H), 7.61- 7.55 (m, 3H), 7.55-7.43 (m, 4H), 7.37- 7.28 (m, 1H), 7.17 (t, J = 7.0 Hz, 1H), 6.89 (s, 1H), 4.36 (s, 2H), 3.98-3.87 (m, 1H), 3.63 (s, 3H), 2.09 (s, 3H), 1.22 (d, J = 6.9 Hz, 6H). |
| 2 | 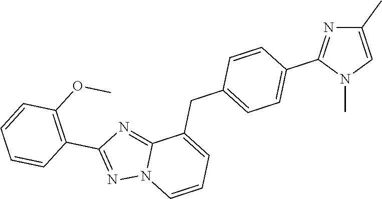 [SMILES: COc1ccccc1-c1nc2c(Cc3ccc(-c4nc(C)cn4C)cc3)cccn2n1] | 409.49 | 410.2 [M + H]⁺ | DMSO-d₆: δ 8.85 (d, J = 6.5 Hz, 1H), 7.94-7.87 (m, 1H), 7.59 (d, J = 8.1 Hz, 2H), 7.55-7.45 (m, 4H), 7.20 (d, J = 8.4 Hz, 1H), 7.16-7.05 (m, 2H), 6.92 (s, 1H), 4.37 (s, 2H), 3.85 (s, 3H), 3.64 (s, 3H), 2.10 (s, 3H). |
| 3 | 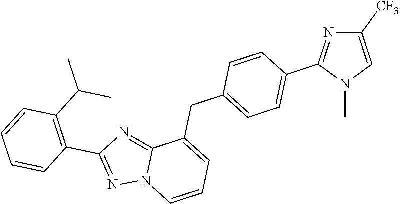 [SMILES: CC(C)c1ccccc1-c1nc2c(Cc3ccc(-c4nc(C(F)(F)F)cn4C)cc3)cccn2n1] | 475.52 | 476.2 [M + H]⁺ | DMSO-d₆: δ 8.87 (d, J = 6.0 Hz, 1H), 7.90 (s, 1H), 7.82 (dd, J = 7.7, 1.1 Hz, 1H), 7.65 (d, J = 8.3 Hz, 2H), 7.62- 7.55 (m, 3H), 7.53-7.43 (m, 2H), 7.32 (td, J = 7.7, 1.4 Hz, 1H), 7.17 (t, J = 7.0 Hz, 1H), 4.39 (s, 2H), 3.93 (dt, J = 13.7, 6.8 Hz, 1H), 3.75 (s, 3H), 1.22 (d, J = 6.9 Hz, 6H). |
| 4 | 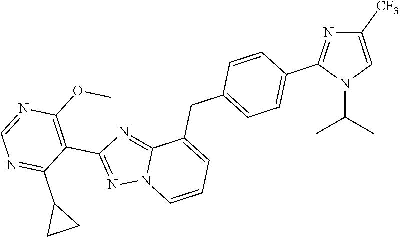 [SMILES: COc1ncnc(C2CC2)c1-c1nc2c(Cc3ccc(-c4nc(C(F)(F)F)cn4C(C)C)cc3)cccn2n1] | 533.36 | 534.2 [M + H]⁺ | DMSO-d₆: δ 8.91 (d, J = 6.4 Hz, 1H), 8.71 (s, 1H), 8.15 (s, 1H), 7.61-7.52 (m, 3H), 7.49 (d, J = 8.2 Hz, 2H), 7.22 (t, J = 7.0 Hz, 1H), 4.50-4.43 (m, 1H), 4.41 (s, 2H), 3.88 (s, 3H), 1.92-1.85 (m, 1H), 1.38 (d, J = 6.6 Hz, 6H), 1.10-1.04 (m, 2H), 0.95-0.84 (m, 2H). |
| 5 | 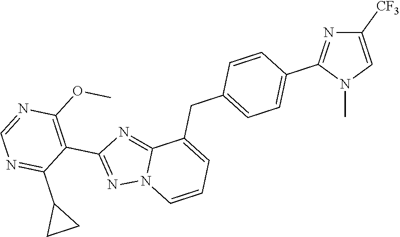 [SMILES: COc1ncnc(C2CC2)c1-c1nc2c(Cc3ccc(-c4nc(C(F)(F)F)cn4C)cc3)cccn2n1] | 505.51 | 506.2 [M + H]⁺ | DMSO-d₆: δ 8.91 (d, J = 6.6 Hz, 1H), 8.73 (s, 1H), 7.91 (s, 1H), 7.64 (d, J = 8.2 Hz, 2H), 7.58-7.51 (m, 3H), 7.22 (t, J = 7.0 Hz, 1H), 4.40 (s, 2H), 3.88 (s, 3H), 3.75 (s, 3H), 1.95-1.85 (m, 1H), 1.11-1.03 (m, 2H), 0.94-0.87 (m, 2H). |
| 6 | 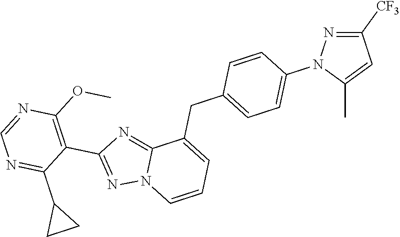 [SMILES: COc1ncnc(C2CC2)c1-c1nc2c(Cc3ccc(-n4nc(C(F)(F)F)cc4C)cc3)cccn2n1] | 505.51 | 506.2 [M + H]⁺ | DMSO-d₆: δ 8.92 (d, J = 6.7 Hz, 1H), 8.73 (s, 1H), 7.63-7.55 (m, 3H), 7.49 (d, J = 8.4 Hz, 2H), 7.23 (t, J = 7.0 Hz, 1H), 6.74 (s, 1H), 4.42 (s, 2H), 3.88 (s, 3H), 2.31 (s, 3H), 1.93-1.85 (m, 1H), 1.11-1.04 (m, 2H), 0.97-0.89 (m, 2H). |
| 7 | 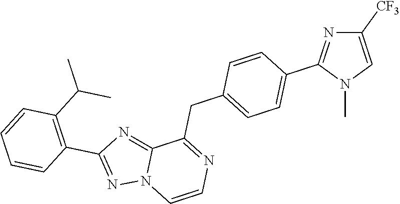 [SMILES: CC(C)c1ccccc1-c1nc2c(Cc3ccc(-c4nc(C(F)(F)F)cn4C)cc3)nccn2n1] | 476.51 | / | / |
| 8 | 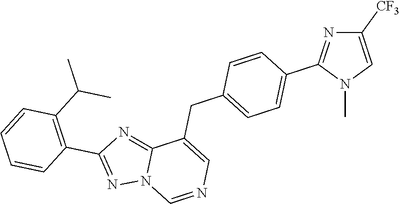 [SMILES: CC(C)c1ccccc1-c1nc2c(Cc3ccc(-c4nc(C(F)(F)F)cn4C)cc3)cncn2n1] | 476.51 | / | / |
| 9 | 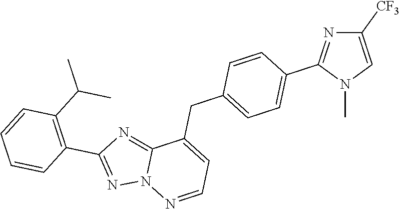 [SMILES: CC(C)c1ccccc1-c1nc2c(Cc3ccc(-c4nc(C(F)(F)F)cn4C)cc3)ccnn2n1] | 476.51 | 477.2 [M + H]⁺ | DMSO-d₆: δ 8.68 (d, J = 4.7 Hz, 1H), 7.91 (s, 1H), 7.87 (d, J = 8.0 Hz, 1H), 7.69 (d, J = 8.1 Hz, 2H), 7.63 (d, J = 8.1 Hz, 2H), 7.56-7.46 (m, 3H), 7.35 (t, J = 7.3 Hz, 1H), 4.48 (s, 2H), 3.98- 3.89 (m, 1H), 3.76 (s, 3H), 1.24 (d, J = 6.8 Hz, 6H). |

### Example 10

### 2-(2-isopropylphenyl)-5-(4-(1-methyl-4-(trifluoromethyl)-1H-imidazol-2-yl)benzyl)-[1,2,4]triazolo[1,5-a]pyridine

a) Preparation of carbamic acid, [[(6-methyl-2-pyridinyl)amino]thioxomethyl]-, ethyl ester: To a mixture of 6-methylpyridin-2-amine (5.0 g, 46.2 mmol) in 1, 4-dioxane (50 mL) was added O-ethyl carbonisothiocyanatidate (6.0 g, 45.8 mmol). The mixture was stirred at room temperature for overnight. The resulting mixture was concentrated under vacuum and washed with EtOAc (30 mL) to give the title compound (11 g. yellow solid, yield 99.6%). MS (ESI, m/z): 240.1 [M+H]⁺.

b) Preparation of 5-methyl-[1,2,4]triazolo[1,5-a]pyridin-2-amine: To a mixture of carbamic acid. [[(6-methyl-2-pyridinyl)amino]thioxomethyl]-, ethyl ester (11.0 g, 46.0 mmol) in EtOH (50 mL) and MeOH (50 mL) was added NH₂OH·HCl (16.0 g, 231.0 mmol) and DIEA (17.8 g, 137.7 mmol). The mixture was stirred at room temperature for overnight. Then the mixture was concentrated under vacuum and washed with EtOAc (30 mL) to give the title compound (4 g. off-white solid, yield 58.8%). MS (ESI, m/z): 149.1 [M+H]⁺. ¹H NMR (400 MHz, DMSO-d₆): δ 7.38-7.32 (m, 1H), 7.20 (d, J=8.8 Hz, 1H), 7.79-7.72 (m, 1H), 5.96 (brs, 2H), 2.46 (s, 3H).

c) Preparation of 2-chloro-5-methyl-[1,2,4]triazolo[1,5-a]pyridine: To a solution of 5-methyl-[1,2,4]triazolo[1,5-a]pyridin-2-amine (1.0 g, 6.8 mmol) in ACN (20 mL) was added NaNO₂ (559.2 mg, 8.1 mmol) at 0° C. Then con. HCl (1 mL) was added dropwise, and the reaction mixture was stirred at 0° C. for 15 min. Then additional con. HCl was added until the mixture solid was dissolved. The resulting mixture was stirred at 80° C. for 2 h. The mixture was added to ice-water and extracted with DCM (50 mL×3), and the combined organic layers were washed with brine (10 mL), dried over Na₂SO₄, filtered and concentrated in vacuum, and then purified by silica-gel chromatography (hexane:EtOAc=20:1) to give the title compound (480 mg, off white solid, yield 42.4%). MS (ESI, m/z): 168.1 [M+H]⁺. ¹H NMR (400 MHz, DMSO-d₆): δ 7.68-7.62 (m, 2H), 7.16-7.12 (m, 1H), 2.66 (s, 3H).

d) Preparation of 5-(bromomethyl)-2-chloro-[1,2,4]triazolo[1,5-a]pyridine: To a mixture of 2-chloro-5-methyl-[1,2,4]triazolo[1,5-a]pyridine (480 mg, 2.8 mmol) in CCl₄ (10 mL) was added NBS (613.8 mg, 3.4 mmol) and AIBN (47 mg, 0.3 mmol). The mixture was stirred at reflux for overnight under nitrogen atmosphere. The resulting mixture was quenched with water (50 mL) and extracted with EtOAc (30 mL×3), the organic layers were dried over MgSO₄, filtered and concentrated. The concentrated residue was purified by silica-gel chromatography (hexane:EtOAc=10:1) to give the title compound (460 mg, white solid, yield 64.9%). MS (ESI, m/z): 246.0 [M+H]⁺. ¹H NMR (400 MHz, DMSO-d₆): δ 7.86-7.82 (m, 1H), 7.78-7.74 (m, 1H), 7.48-7.46 (m, 1H), 5.07 (s, 2H).

e) Preparation of 2-chloro-5-(4-(1-methyl-4-(trifluoromethyl)-1H-imidazol-2-yl)benzyl)-[1,2,4]triazolo[1,5-a]pyridine: To a mixture of 5-(bromomethyl)-2-chloro-[1,2,4]triazolo[1,5-a]pyridine (230 mg, 0.94 mmol) in dioxane (5 mL) was added (4-(1-methyl-4-(trifluoromethyl)-1H-imidazol-2-yl)phenyl)boronic acid (255 mg, 0.94 mmol), Pd(PPh₃)₂Cl₂ (66 mg, 0.09 mmol) and Na₂CO₃ (300 mg, 2.8 mmol). The mixture was stirred at 60° C. for 4 h under nitrogen atmosphere. The resulting mixture was quenched with water (50 mL) and extracted with EtOAc (30 mL×3), the organic layers were dried over Na₂SO₄, filtered and concentrated. The concentrated residue was purified by silica-gel chromatography (hexane:EtOAc=2:1) to give the title compound (250 mg, yellow solid, yield 68.5%). MS (ESI, m/z): 246.0 [M+H]⁺.

f) Preparation of 2-(2-isopropylphenyl)-5-(4-(1-methyl-4-(trifluoromethyl)-1H-imidazol-2-yl)benzyl)-[1,2,4]triazolo[1,5-a]pyridine: To a mixture of 2-chloro-5-(4-(1-methyl-4-(trifluoromethyl)-1H-imidazol-2-yl)benzyl)-[1,2,4]triazolo[1,5-a]pyridine (140 mg, 0.35 mmol) in toluene (5 mL) was added (2-isopropylphenyl)boronic acid (56 mg, 0.53 mmol), cataCXium A Pd G3 (26 mg, 0.03 mmol) and Cs₂CO₃ (349 mg, 1.0 mmol). The mixture was stirred with microwave reaction at 120° C. under nitrogen atmosphere for 4 h. The resulting mixture was quenched with water (30 mL) and extracted with EtOAc (20 mL×3), and the organic layers concentrated. The crude product was purified by prep-HPLC (ACN/0.1% FA aq=10%) to give the target compound (11.3 mg, white solid, yield 6.6%).

The following compounds of Examples 11-17 were prepared using a synthesis method similar to that described in Example 1 and 10.

| | | | LC-MS | |
|---|---|---|---|---|
| Example | Compound | MW | (ESI) | ¹H NMR (400 MHz) |
| 10 | 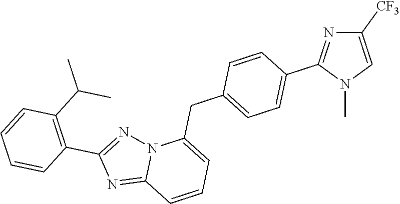 [SMILES: CC(C)c1ccccc1-c1nc2cccc(Cc3ccc(-c4nc(C(F)(F)F)cn4C)cc3)n2n1] | 475.52 | 476.2 [M + H]⁺ | DMSO-d₆: δ 7.91 (s, 1H), 7.85 (d, J = 8.3 Hz, 1H), 7.82 (d, J = 8.0 Hz, 1H), 7.74- 7.65 (m, 3H), 7.54 (d, J = 8.0 Hz, 2H), 7.56-7.42 (m, 2H), 7.32 (t, J = 7.3 Hz, 1H), 7.17 (d, J = 7.0 Hz, 1H), 4.62 (s, 2H), 3.96-3.87 (m, 1H), 3.76 (s, 3H), 1.19 (d, J = 6.8 Hz, 6H). |
| 11 | 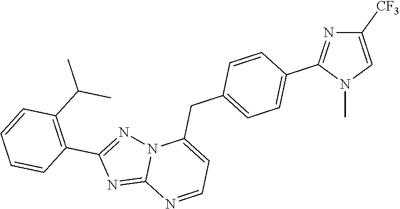 [SMILES: CC(C)c1ccccc1-c1nc2nccc(Cc3ccc(-c4nc(C(F)(F)F)cn4C)cc3)n2n1] | 476.51 | / | / |
| 12 | 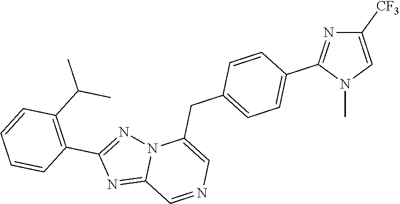 [SMILES: CC(C)c1ccccc1-c1nc2cncc(Cc3ccc(-c4nc(C(F)(F)F)cn4C)cc3)n2n1] | 476.51 | / | / |
| 13 | 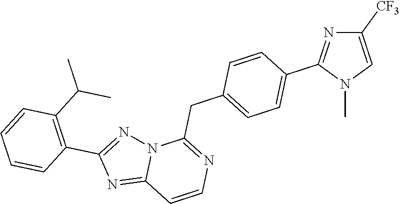 [SMILES: CC(C)c1ccccc1-c1nc2ccnc(Cc3ccc(-c4nc(C(F)(F)F)cn4C)cc3)n2n1] | 476.51 | / | / |
| 14 | 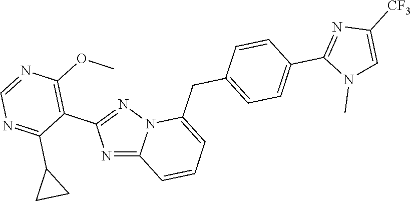 [SMILES: COc1ncnc(C2CC2)c1-c1nc2cccc(Cc3ccc(-c4nc(C(F)(F)F)cn4C)cc3)n2n1] | 505.51 | 506. [M + H]⁺ | DMSO-d₆: δ 8.72 (s, 1H), 7.92 (s, 1H), 7.83 (d, J = 8.8 Hz, 1H), 7.75-7.63 (m, 3H), 7.54 (d, J = 7.2 Hz, 2H), 7.11 (d, J = 6.7 Hz, 1H), 4.61 (s, 2H), 3.87 (s, 3H), 3.77 (s, 3H), 1.90-1.81 (m, 1H), 1.11-1.01 (m, 2H), 0.90-0.84 (m, 2H). |
| 15 | 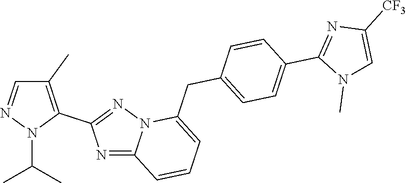 [SMILES: Cc1cnn(C(C)C)c1-c1nc2cccc(Cc3ccc(-c4nc(C(F)(F)F)cn4C)cc3)n2n1] | 479.51 | 480.3 [M + H]⁺ | DMSO-d₆ (300 MHz): δ 7.92-7.83 (m, 2H), 7.80-7.63 (m, 3H), 7.53 (d, J = 7.8 Hz, 2H), 7.43 (s, 1H), 7.26 (d, J = 7.2 Hz, 1H), 5.64-7.48 (m, 1H), 4.62 (s, 2H), 3.75 (s, 3H), 2.27 (s, 3H), 1.39 (d, J = 6.4 Hz, 6H). |
| 16 | 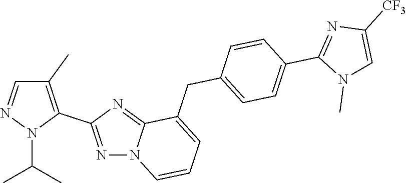 [SMILES: Cc1cnn(C(C)C)c1-c1nc2c(Cc3ccc(-c4nc(C(F)(F)F)cn4C)cc3)cccn2n1] | 479.51 | 480.2 [M + H]⁺ | DMSO-d₆ (300 MHz): δ 8.91 (d, J = 6.0 Hz, 1H), 7.90 (d, J = 1.2 Hz, 1H), 7.70- 7.66 (m, 3H), 7.58 (d, J = 9.0 Hz, 2H), 7.45 (s, 1H), 7.22 (t, J = 7.0 Hz, 1H), 5.63- 5.52 (m, 1H), 4.39 (s, 2H), 3.74 (s, 3H), 2.30 (s, 3H), 1.43 (d, J = 6.6 Hz, 6H). |
| 17 | 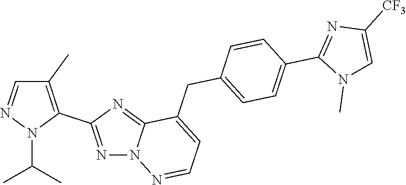 [SMILES: Cc1cnn(C(C)C)c1-c1nc2c(Cc3ccc(-c4nc(C(F)(F)F)cn4C)cc3)ccnn2n1] | 480.50 | / | / |

### Example 18

### 2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-a]pyrazine

a) Preparation of 3-(4-(1-methyl-4-(trifluoromethyl)-1H-imidazol-2-yl)benzyl)pyrazin-2-amine: A solution of Zn (202.0 mg, 3.1 mmol), I₂ (20.0 mg, 0.08 mmol) in DMF (10 mL) was stirred at 30° C. for 10 min under N₂. Then TMSCl (8.0 mg, 0.08 mmol) was added and stirred at 30° C. for 45 min, and then 2-(4-(bromomethyl)phenyl)-1-methyl-4-(trifluoromethyl)-1H-imidazole (95 mg, 0.26 mmol) in DMF (1 mL) was added and stirred at 45° C. for 1 h. The mixture was added to a system of Pd(PPh₃)₄ (30.0 mg, 0.026 mmol) and 3-bromopyrazin-2-amine (45 mg, 0.26 mmol), which was stirred at 60° C. for 2 h under N₂. After completion, the mixture was cooled down to room temperature, and the mixture was added water (100 mL) and extracted with EA (40 mL×3). The combined organic phase was washed with brine, dried over anhydrous sodium sulfate and concentrated under reduced pressure. The residue was purified by Prep-TLC: (DCM:MeOH=10:1) to afford the title compound (40.0 mg, white solid, yield: 46%). MS(ESI): 334.25 [M+H]⁺.

b) Preparation of 1,2-diamino-3-(4-(1-methyl-4-(trifluoromethyl)-1H-imidazol-2-yl)benzyl)pyrazin-1-ium·2,4,6-trimethylbenzenesulfonate: A solution of 3-(4-(1-methyl-4-(trifluoromethyl)-1H-imidazol-2-yl)benzyl)pyrazin-2-amine (40 mg, 0.12 mmol) and 0-(mesitylsulfonyl)hydroxylamine (51.6 mg, 0.24 mmol) in DCM (5 mL) was stirred at room temperature for 16 h. After completion, the solvent was evaporated to give the title compound (50.5 mg, yellow solid, crude) used for the next step directly. MS(ESI): 349.30 [M+H]⁺.

c) Preparation of 2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-a]pyrazine: To a solution of 1,2-diamino-3-(4-(1-methyl-4-(trifluoromethyl)-1H-imidazol-2-yl)benzyl)pyrazin-1-ium·2,4,6-trimethylbenzenesulfonate (50.5 mg, 0.12 mmol) in dioxane (5 mL) was added 4-cyclopropyl-6-methoxypyrimidine-5-carbaldehyde (43 mg, 0.24 mmol) and Cs₂CO₃ (117 mg, 0.36 mmol). The mixture was stirred at 90° C. for 2 h. After completion, the mixture was added water (50 mL) and extracted with EA (20 mL×3). The combined organic phase was washed with brine, dried over anhydrous sodium sulfate and concentrated under reduced pressure, the residue was purified by Prep-HPLC to afford the target compound (5.0 mg, white solid, 2 steps yield: 9%).

The following compounds of Examples 19-47 were prepared using a synthesis method similar to that described in Example 18.

| | | | LC-MS | |
|---|---|---|---|---|
| Example | Compound | MW | (ESI) | ¹H NMR (400 MHz) |
| 18 | 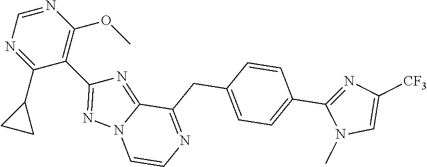 [SMILES: COc1ncnc(C2CC2)c1-c1nc2c(Cc3ccc(-c4nc(C(F)(F)F)cn4C)cc3)nccn2n1] | 506.48 | 507.35 [M + H]⁺ | CDCl₃: δ 8.67 (s, 1H), 8.47 (d, J = 4.0 Hz, 1H), 8.12 (d, J = 4.0 Hz, 1H), 7.61 (d, J = 8.0 Hz, 2H), 7.54 (d, J = 8.0 Hz, 2H), 7.27 (s, 1H), 4.68 (s, 2H), 3.95 (s, 3H), 3.72 (s, 3H), 1.92-1.90 (m, 1H), 1.26-1.24 (m, 2H), 0.94-0.92 (m, 2H). |
| 19 | 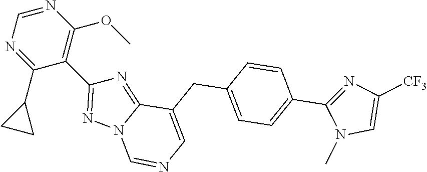 [SMILES: COc1ncnc(C2CC2)c1-c1nc2c(Cc3ccc(-c4nc(C(F)(F)F)cn4C)cc3)cncn2n1] | 506.48 | 507.25 [M + H]⁺ | CDCl₃: δ 9.35 (s, 1H), 8.67 (s, 1H), 7.99 (s, 1H), 7.59-7.57 (m, 2H), 7.51-7.49 (m, 2H), 7.29 (s, 1H), 4.41 (s, 2H), 3.95 (s, 3H), 3.74 (s, 3H), 1.97-1.93 (m, 1H), 1.26-1.24 (m, 2H), 0.94-0.92 (m, 2H). |
| 20 | 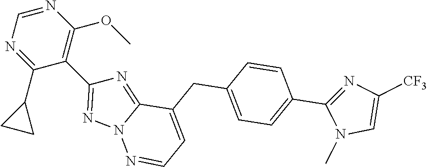 [SMILES: COc1ncnc(C2CC2)c1-c1nc2c(Cc3ccc(-c4nc(C(F)(F)F)cn4C)cc3)ccnn2n1] | 506.48 | / | / |
| 21 | 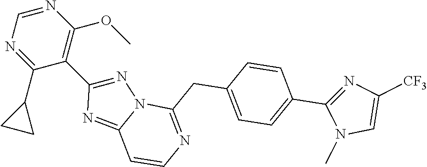 [SMILES: COc1ncnc(C2CC2)c1-c1nc2ccnc(Cc3ccc(-c4nc(C(F)(F)F)cn4C)cc3)n2n1] | 506.48 | 507.15 [M + H]⁺ | CD₃OD: δ 8.64 (s, 1H), 8.32 (d, J = 6.4 Hz, 1H), 7.72 (d, J = 6.3 Hz, 1H), 7.66 (s, 1H), 7.63 (d, J = 8.1 Hz, 2H), 7.59 (d, J = 8.1 Hz, 2H), 4.79 (s, 2H), 3.93 (s, 3H), 3.74 (s, 3H), 1.94-1.90 (m, 1H), 1.21-1.14 (m, 2H), 0.93- 0.90 (m, 2H). |
| 22 | 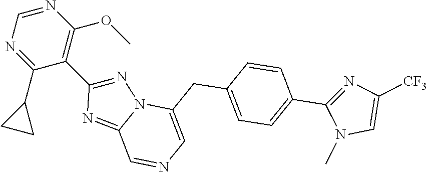 [SMILES: COc1ncnc(C2CC2)c1-c1nc2cncc(Cc3ccc(-c4nc(C(F)(F)F)cn4C)cc3)n2n1] | 506.48 | 507.10 [M + H]⁺ | DMSO-d₆: δ 9.37 (s, 1H), 8.71 (s, 1H), 8.26 (s, 1H), 7.88 (s, 1H), 7.64 (d, J = 7.9 Hz, 2H), 7.53 (d, J = 8.0 Hz, 2H), 4.60 (s, 2H), 3.84 (s, 3H), 3.72 (s, 3H), 1.91-1.76 (m, 1H), 1.07-0.97 (m, 2H), 0.88-0.78 (m, 2H). |
| 23 | 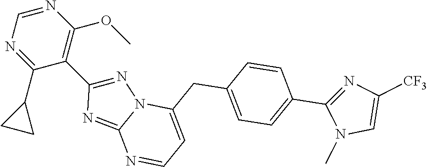 [SMILES: COc1ncnc(C2CC2)c1-c1nc2nccc(Cc3ccc(-c4nc(C(F)(F)F)cn4C)cc3)n2n1] | 506.48 | 507.15 [M + H]⁺ | DMSO-d₆: δ 8.84 (d, J = 4.5 Hz, 1H), 8.71 (s, 1H), 7.90 (s, 1H), 7.67 (d, J = 8.1 Hz, 2H), 7.55 (d, J = 7.9 Hz, 2H), 7.16 (d, J = 4.5 Hz, 1H), 4.63 (s, 2H), 3.86 (s, 3H), 3.74 (s, 3H), 1.92-1.90 (m, 1H), 1.07-1.02 (m, 2H), 0.95-0.90 (m, 2H). |
| 24 | 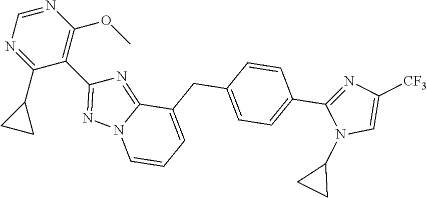 [SMILES: COc1ncnc(C2CC2)c1-c1nc2c(Cc3ccc(-c4nc(C(F)(F)F)cn4C4CC4)cc3)cccn2n1] | 531.54 | 532.20 [M + H]⁺ | CDCl₃: δ 8.66 (s, 1H), 8.52 (d, J = 8.0 Hz, 1H), 7.78 (d, J = 8.0 Hz, 2H), 7.44 (d, J = 8.0 Hz, 2H), 7.31 (s, 1H), 7.14 (d, J = 8.0 Hz, 1H), 6.97 (t, J = 4.8 Hz, 1H), 4.46 (s, 2H), 3.95 (s, 3H), 3.48-3.46 (m, 1H), 1.99-1.93 (m, 1H), 1.23-1.20 (m, 2H), 1.09-1.04 (m, 2H), 0.92-0.88 (m, 4H). |
| 25 | 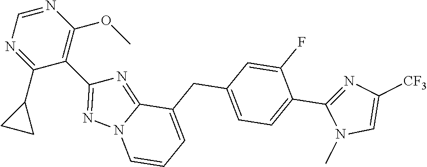 [SMILES: COc1ncnc(C2CC2)c1-c1nc2c(Cc3ccc(-c4nc(C(F)(F)F)cn4C)c(F)c3)cccn2n1] | 523.50 | 524.20 [M + H]⁺ | CDCl₃: δ 8.66 (s, 1H), 8.55 (d, J = 8.0 Hz, 1H), 7.57-7.53 (m, 1H), 7.33 (s, 1H), 7.28- 7.26 (m, 2H), 7.21 (s, 1H), 7.0 (t, J = 8.0 Hz, 1H), 4.45 (s, 2H), 3.95 (s, 3H), 3.62 (s, 3H), 1.97-1.93 (m, 1H), 1.26-1.24 (m, 2H), 0.94- 0.92 (m, 2H). |
| 26 | 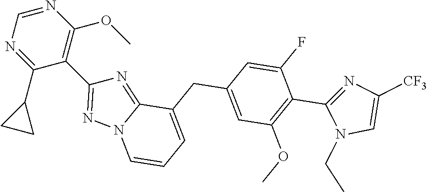 [SMILES: CCn1cc(C(F)(F)F)nc1-c1c(F)cc(Cc2cccn3nc(-c4c(OC)ncnc4C4CC4)nc23)cc1OC] | 567.55 | 568.20 [M + H]⁺ | CDCl₃: δ 8.66 (s, 1H), 8.55 (d, J = 6.7 Hz, 1H), 7.38 (s, 1H), 7.22 (d, J = 7.2 Hz, 1H), 7.01 (t, J = 7.0 Hz, 1H), 6.86-6.78 (m, 2H), 4.42 (s, 2H), 3.94 (s, 3H), 3.77 (q, J = 7.6 Hz, 2H), 3.74 (s, 3H), 1.90 (ddd, J = 12.8, 8.3, 4.8 Hz, 1H), 1.33 (t, J = 7.3 Hz, 3H), 1.25-1.20 (m, 2H), 0.95-0.89 (m, 2H). |
| 27 | 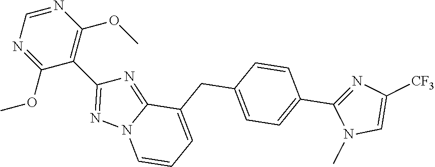 [SMILES: COc1ncnc(OC)c1-c1nc2c(Cc3ccc(-c4nc(C(F)(F)F)cn4C)cc3)cccn2n1] | 495.47 | 496.90 [M + H]⁺ | CDCl₃: δ 8.53 (s, 1H), 8.49 (d, J = 5.3 Hz, 1H), 7.58 (d, J = 7.6 Hz, 2H), 7.45 (d, J = 7.8 Hz, 2H), 7.29 (s, 1H), 7.11-7.04 (m, 1H), 6.97-6.88 (m, 1H), 4.46 (s, 2H), 3.97 (s, 6H), 3.75 (s, 3H). |
| 28 | 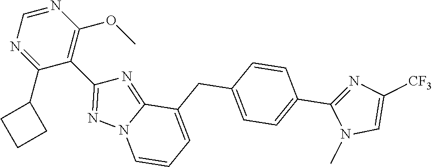 [SMILES: COc1ncnc(C2CCC2)c1-c1nc2c(Cc3ccc(-c4nc(C(F)(F)F)cn4C)cc3)cccn2n1] | 519.53 | 520.20 [M + H]⁺ | CDCl₃: δ 8.87 (s, 1H), 8.51 (d, J = 6.8 Hz, 1H), 7.58 (d, J = 8.0 Hz, 2H), 7.46 (d, J = 7.6 Hz, 2H), 7.29 (s, 1H), 7.18 (d, J = 6.8 Hz, 1H), 7.0-6.96 (t, J = 4.8 Hz, 1H), 4.44 (s, 2H), 3.95 (s, 3H), 3.75 (s, 3H), 3.61-3.56 (m, 1H), 2.45-2.40 (m, 2H), 1.99-1.96 (m, 2H), 1.83-1.78 (m, 2H). |
| 29 | 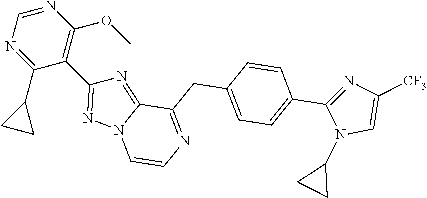 [SMILES: COc1ncnc(C2CC2)c1-c1nc2c(Cc3ccc(-c4nc(C(F)(F)F)cn4C4CC4)cc3)nccn2n1] | 532.53 | 533.20 [M + H]⁺ | CDCl₃: δ 8.68 (s, 1H), 8.47 (d, J = 4.0 Hz, 1H), 8.13 (d, J = 4.0 Hz, 1H), 7.75 (d, J = 8.0 Hz, 2H), 7.59 (d, J = 8.0 Hz, 2H), 7.29 (s, 1H), 4.68 (s, 2H), 3.95 (s, 3H), 3.48-3.44 (m, 1H), 1.97-1.90 (m, 1H), 1.25-1.20 (m, 2H), 1.05-1.03 (m, 2H), 0.92-0.90 (m, 2H), 0.88-0.86 (m, 2H). |
| 30 | 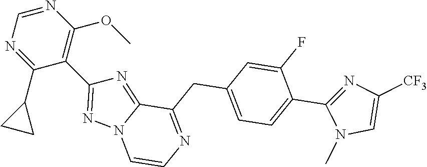 [SMILES: COc1ncnc(C2CC2)c1-c1nc2c(Cc3ccc(-c4nc(C(F)(F)F)cn4C)c(F)c3)nccn2n1] | 524.48 | 525.20 [M + H]⁺ | CDCl₃: δ 8.68 (s, 1H), 8.49 (d, J = 4.0 Hz, 1H), 8.13 (d, J = 4.0 Hz, 1H), 7.52 (t, J = 8.0 Hz, 1H), 7.41 (s, 1H), 7.39-7.37 (m, 1H), 7.31 (s, 1H), 4.67 (s, 2H), 3.95 (s, 3H), 3.60 (s, 3H), 1.94-1.92 (m, 1H), 1.26-1.24 (m, 2H), 0.96-0.93 (m, 2H). |
| 31 | 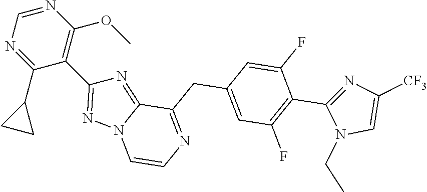 [SMILES: CCn1cc(C(F)(F)F)nc1-c1c(F)cc(Cc2nccn3nc(-c4c(OC)ncnc4C4CC4)nc23)cc1F] | 556.50 | 557.15 [M + H]⁺ | CDCl₃: δ 8.68 (s, 1H), 8.52 (d, J = 4.0 Hz, 1H), 8.14 (d, J = 4.0 Hz, 1H), 7.40 (s, 1H), 7.23-7.22 (m, 2H), 4.65 (s, 2H), 3.95 (s, 3H), 3.85-3.80 (m, 2H), 1.97-1.93 (m, 1H), 1.36 (t, J = 8.0 Hz, 3H), 1.26-1.24 (m, 2H), 0.97- 0.95 (m, 2H). |
| 32 | 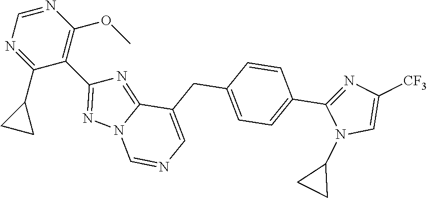 [SMILES: COc1ncnc(C2CC2)c1-c1nc2c(Cc3ccc(-c4nc(C(F)(F)F)cn4C4CC4)cc3)cncn2n1] | 532.53 | 533.20 [M + H]⁺ | DMSO-d₆: δ 9.77 (s, 1H), 8.71 (s, 1H), 8.27 (s, 1H), 7.88 (s, 1H), 7.79 (d, J = 8.2 Hz, 2H), 7.51 (d, J = 8.3 Hz, 2H), 4.34 (s, 2H), 3.84 (s, 3H), 3.73-3.62 (m, 1H), 1.92-1.81 (m, 1H), 1.09-1.00 (m, 2H), 0.97-0.90 (m, 2H), 0.90-0.82 (m, 4H). |
| 33 | 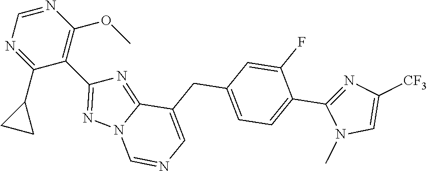 [SMILES: COc1ncnc(C2CC2)c1-c1nc2c(Cc3ccc(-c4nc(C(F)(F)F)cn4C)c(F)c3)cncn2n1] | 524.48 | 525.20 [M + H]⁺ | CDCl₃: δ 9.37 (s, 1H), 8.68 (s, 1H), 8.05 (s, 1H), 7.56 (t, J = 7.9 Hz, 1H), 7.32 (d, J = 8.4 Hz, 2H), 7.28 (d, J = 4.1 Hz, 1H), 4.39 (s, 2H), 3.95 (s, 3H), 3.61 (d, J = 1.9 Hz, 3H), 1.96-1.88 (m, 1H), 1.28-1.21 (m, 2H), 0.99-0.89 (m, 2H). |
| 34 | 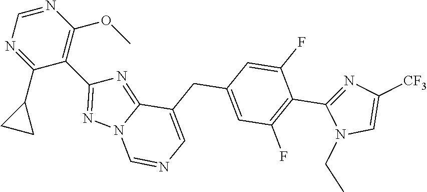 [SMILES: CCn1cc(C(F)(F)F)nc1-c1c(F)cc(Cc2cncn3nc(-c4c(OC)ncnc4C4CC4)nc23)cc1F] | 556.50 | 557.35 [M + H]⁺ | CDCl₃: δ 9.38 (s, 1H), 8.67 (s, 1H), 8.09 (s, 1H), 7.41 (s, 1H), 7.12 (d, J = 8.0 Hz, 2H), 4.37 (s, 2H), 3.95 (s, 3H), 3.85-3.80 (m, 2H), 1.97-1.93 (m, 1H), 1.36 (t, J = 8.0 Hz, 3H), 1.26-1.24 (m, 2H), 0.97-0.95 (m, 2H). |
| 35 | 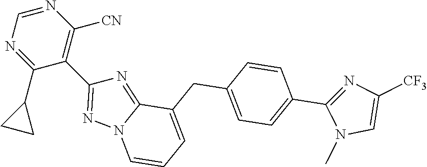 [SMILES: Cn1cc(C(F)(F)F)nc1-c1ccc(Cc2cccn3nc(-c4c(C#N)ncnc4C4CC4)nc23)cc1] | 500.49 | / | / |
| 36 | 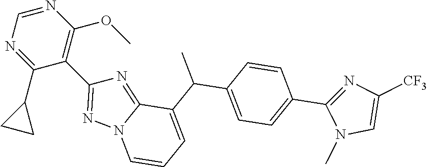 [SMILES: COc1ncnc(C2CC2)c1-c1nc2c(C(C)c3ccc(-c4nc(C(F)(F)F)cn4C)cc3)cccn2n1] | 519.53 | / | / |
| 37 | 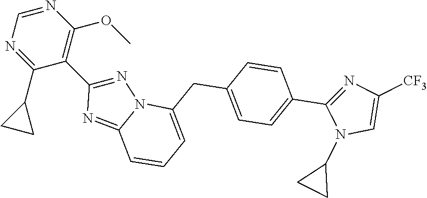 [SMILES: COc1ncnc(C2CC2)c1-c1nc2cccc(Cc3ccc(-c4nc(C(F)(F)F)cn4C4CC4)cc3)n2n1] | 531.54 | 532.20 [M + H]⁺ | CDCl₃: δ 8.66 (s, 1H), 7.83 (d, J = 8.0 Hz, 2H), 7.72 (d, J = 8.0 Hz, 1H), 7.49 (d, J = 8.0 Hz, 1H), 7.45 (d, J = 8.0 Hz, 2H), 7.33 (s, 1H), 6.66 (d, J = 8.0 Hz, 1H), 4.59 (s, 2H), 3.95 (s, 3H), 3.50-3.46 (s, 1H), 1.97-1.93 (m, 1H), 1.23-1.20 (m, 2H), 1.09-1.04 (m, 2H), 0.92-0.88 (m, 4H). |
| 38 | 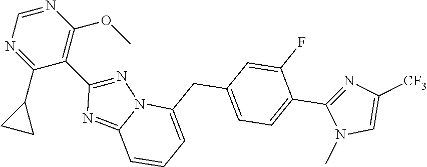 [SMILES: COc1ncnc(C2CC2)c1-c1nc2cccc(Cc3ccc(-c4nc(C(F)(F)F)cn4C)c(F)c3)n2n1] | 523.50 | 524.15 [M + H]⁺ | CDCl₃: δ 8.67 (s, 1H), 7.75 (d, J = 8.0 Hz, 1H), 7.59 (t, J = 8.0 Hz, 1H), 7.51 (t, J = 8.0 Hz, 1H), 7.34 (s, 1H), 7.30-7.24 (m, 2H), 6.76 (d, J = 4.0 Hz, 1H), 4.59 (s, 2H), 3.95 (s, 3H), 3.63 (s, 3H), 1.97-1.93 (m, 1H), 1.23-1.20 (m, 2H), 0.92-0.90 (m, 2H). |
| 39 | 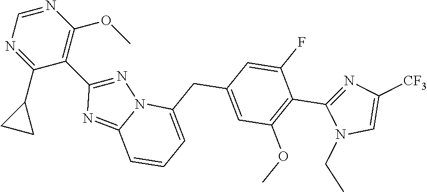 [SMILES: CCn1cc(C(F)(F)F)nc1-c1c(F)cc(Cc2cccc3nc(-c4c(OC)ncnc4C4CC4)nn23)cc1OC] | 567.55 | 568.20 [M + H]⁺ | CDCl₃: δ 8.67 (s, 1H), 7.75 (d, J = 8.9 Hz, 1H), 7.52 (dd, J = 8.8, 7.2 Hz, 1H), 7.39 (s, 1H), 6.83 (d, J = 9.2 Hz, 1H), 6.80 (s, 1H), 6.74 (d, J = 7.0 Hz, 1H), 4.55 (s, 2H), 3.94 (s, 3H), 3.83-3.72 (m, 5H), 1.91 (td, J = 8.0, 4.3 Hz, 1H), 1.34 (t, J = 7.4 Hz, 3H), 1.25- 1.19 (m, 2H), 0.94-0.87 (m, 2H). |
| 40 | 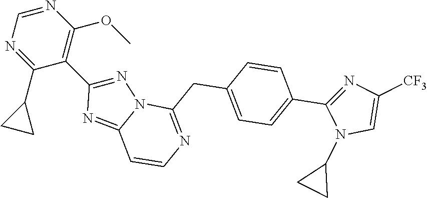 [SMILES: COc1ncnc(C2CC2)c1-c1nc2ccnc(Cc3ccc(-c4nc(C(F)(F)F)cn4C4CC4)cc3)n2n1] | 532.53 | 533.70 [M + H]⁺ | DMSO-d₆: δ 8.71 (s, 1H), 8.29 (d, J = 6.4 Hz, 1H), 7.91-7.79 (m, 4H), 7.51 (d, J = 8.2 Hz, 2H), 4.73 (s, 2H), 3.85 (s, 3H), 3.72-3.67 (m, 1H), 1.92-1.86 (m, 1H), 1.06-1.05 (m, 2H), 0.98-0.92 (m, 2H), 0.90-0.84 (m, 4H). |
| 41 | 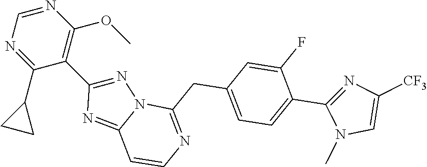 [SMILES: COc1ncnc(C2CC2)c1-c1nc2ccnc(Cc3ccc(-c4nc(C(F)(F)F)cn4C)c(F)c3)n2n1] | 524.48 | 525.25 [M + H]⁺ | DMSO-d₆: δ 8.72 (s, 1H), 8.29 (d, J = 6.4 Hz, 1H), 7.96 (s, 1H), 7.86 (d, J = 6.4 Hz, 1H), 7.52 (t, J = 8.0 Hz, 1H), 7.48 (d, J = 8.4 Hz, 1H), 7.37 (d, J = 8.4 Hz, 1H), 4.77 (s, 2H), 3.86 (s, 3H), 3.57 (s, 3H), 1.96-1.88 (m, 1H), 1.08-1.02 (m, 2H), 0.94-0.84 (m, 2H). |
| 42 | 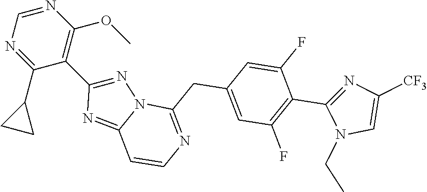 [SMILES: CCn1cc(C(F)(F)F)nc1-c1c(F)cc(Cc2nccc3nc(-c4c(OC)ncnc4C4CC4)nn23)cc1F] | 556.50 | 557.15 [M + H]⁺ | CDCl₃: δ 8.67 (s, 1H), 8.06 (d, J = 9.8 Hz, 2H), 7.54 (d, J = 7.6 Hz, 1H), 7.42 (s, 1H), 6.12 (d, J = 7.7 Hz, 1H), 5.09 (s, 2H), 3.93 (s, 3H), 3.91-3.86 (m, 2H), 2.22-1.98 (m, 1H), 1.37 (t, J = 7.3 Hz, 3H), 1.32-1.20 (m, 4H). |
| 43 | 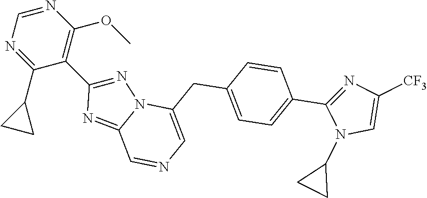 [SMILES: COc1ncnc(C2CC2)c1-c1nc2cncc(Cc3ccc(-c4nc(C(F)(F)F)cn4C4CC4)cc3)n2n1] | 532.53 | 533.20 [M + H]⁺ | DMSO-d₆: δ 9.41 (s, 1H), 8.74 (s, 1H), 8.28 (s, 1H), 7.91 (s, 1H), 7.84 (d, J = 8.2 Hz, 2H), 7.54 (d, J = 8.2 Hz, 2H), 4.63 (s, 2H), 3.87 (s, 3H), 3.74-3.68 (m, 1H), 1.90-1.83 (m, 1H), 1.09-1.02 (m, 2H), 1.00-0.94 (m, 2H), 0.93-0.82 (m, 4H). |
| 44 | 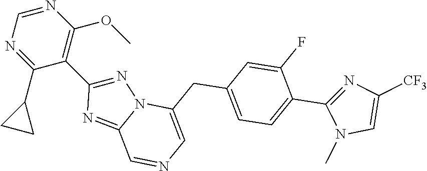 [SMILES: COc1ncnc(C2CC2)c1-c1nc2cncc(Cc3ccc(-c4nc(C(F)(F)F)cn4C)c(F)c3)n2n1] | 524.48 | 525.15 [M + H]⁺ | DMSO-d₆: δ 9.39 (s, 1H), 8.71 (s, 1H), 8.29 (s, 1H), 7.96 (s, 1H), 7.54-7.45 (m, 2H), 7.38 (d, J = 7.6 Hz, 1H), 4.63 (s, 2H), 3.84 (s,3 H), 3.55 (d, J = 1.3 Hz, 3H), 1.93-1.81 (m, 1H), 1.09-0.99 (m, 2H), 0.90-0.79 (m, 2H). |
| 45 | 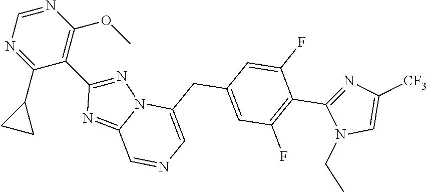 [SMILES: CCn1cc(C(F)(F)F)nc1-c1c(F)cc(Cc2cncc3nc(-c4c(OC)ncnc4C4CC4)nn23)cc1F] | 556.50 | 557.15 [M + H]⁺ | CDCl₃: δ 9.29 (s, 1H), 8.67 (s, 1H), 8.08 (s, 1H), 7.42 (s, 1H), 7.15 (d, J = 8.0 Hz, 2H), 4.56 (s, 2H), 3.94 (s, 3H), 3.83 (q, J = 6.4 Hz, 2H), 1.97-1.88 (m, 1H), 1.36 (t, J = 7.5 Hz, 3H), 0.98-0.88 (m, 2H), 0.52-0.33 (m, 2H). |
| 46 | 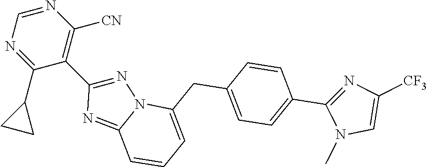 [SMILES: Cn1cc(C(F)(F)F)nc1-c1ccc(Cc2cccc3nc(-c4c(C#N)ncnc4C4CC4)nn23)cc1] | 500.49 | / | / |
| 47 | 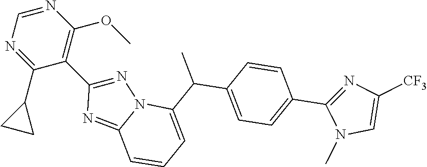 [SMILES: COc1ncnc(C2CC2)c1-c1nc2cccc(C(C)c3ccc(-c4nc(C(F)(F)F)cn4C)cc3)n2n1] | 519.53 | / | / |

### Example 48

### USP1/UAF1 Activity

USP1/UAF1 activity was determined by using ubiquitin-rhodamine110-glycine (Ub-Rho; Boston Biochem) assay. Enzymatic reactions were conducted in an assay buffer (50 mM Tris-HCl, pH 7.8, 0.5 mM EDTA, 100 mM NaCl, 1 mM DTT, 0.01 BSA, and 0.01% Tween-20) that contained 0.1 nM USP1/UAF1. Each individual compound was tested at ten concentrations in the range of 0.0005 to 10 μM. The plates were incubated for 15 min to attain equilibrium, and then the enzymatic reaction was initiated by dispensing 10 μL of Ub-Rho solution (100 nM final concentration). After treating 120 minutes with Ub-Rho solution, the rhodamine fluorescence was acquired using a 480 nm excitation/540 nm emission filter set by using Envision instrument. The inhibition rate of the compound to USP1/UAF1 enzyme activity was calculated according to the following formula.

$$Inhibition ( \% ) = \frac{Flurence Readings of positive control - X}{Flurence Readings of positive control - Flurence Readings of negative control}$$

IC₅₀ value is obtained by fitting the s-shaped dose response curve equation by using XL Fit software. The curve equation is Y=100/(1+10\hat{ }(log C−log IC₅₀)). C is the compound concentration.

Table 1 summarizes the inhibitory effects of compounds on USP1/UAF1 activity (IC₅₀).

TABLE 1

| Example | IC₅₀ (nM) |
|---|---|
| 4 | 66.62 |
| 5 | 39.10 |
| 6 | 460.42 |
| 14 | 30.65 |
| 15 | 17.93 |
| 16 | 15.79 |
| 18 | 11.21 |
| 19 | 138.10 |
| 21 | 16.06 |
| 22 | 146.71 |
| 23 | 175.12 |
| 24 | 11.82 |
| 25 | 11.64 |
| 26 | 39.46 |
| 27 | 77.46 |
| 28 | 25.98 |
| 29 | 34.56 |
| 30 | 41.38 |
| 31 | 57.45 |
| 32 | 111.16 |
| 33 | 94.50 |
| 34 | 242.50 |
| 37 | 9.53 |
| 38 | 5.44 |
| 39 | 18.83 |
| 40 | 19.91 |
| 41 | 34.20 |
| 42 | 731.82 |
| 43 | 38.90 |
| 44 | 78.29 |
| 45 | 99.72 |

Therefore, as determined by the Ubiquitin-rhodamine110-glycine assay, the compounds of this disclosure herein have a good inhibitory effect on USP1/UAF1 enzyme activity.

### Example 49

### Growth Inhibition Assays Against BRCA Mutant Human Breast Cancer MDA-MB-436 Cell Line

The cells were cultured in complete medium (DMEM medium+10% FBS+Insulin+glutathione). When the confluence reached about 80%, cells were digested and gently dispensed from the bottom of the dish with a 1 mL pipette. Cell suspension was collected and centrifuged at 500 rpm for 3 min. The supernatant was discarded, and the cell pellet were re-suspended in complete medium. The cells were seeded into a culture dish at an appropriate proportion, and then cultured in a 5% CO₂ incubator at 37° C. The assay was carried out when the cells were in optimum condition and the confluence was reached 80%. Cells in the logarithmic growth phase were taken to centrifugate, and the culture supernatant was removed. The cells were resuspended in refresh complete medium and counted. The resuspended cells were seeded at 3000/well in a 96-well plate and incubated at 37° C., 5% CO₂ incubator overnight. The compound was prepared as below: 1000× dilution tested compound solution to 40× test compound solution by adding 5 μL 1000× compound solution to 120 μL Medium (25-fold dilution). The solution was mixed by oscillation, 0.1% DMSO was used as the control.

The next day, the 96-well plate inoculated with cells was taken out from the incubator, and the culture supernatant was removed. Then fresh medium of 195 uL/well and 5 μL/well of 40× test compound solution as mentioned above were added into the 96 well plate, respectively. Finally, the plate was incubated for 7 days in a 37° C. 5% CO₂ incubator. The medium containing compound was changed on the fourth day.

CTG Method: After 7 days, 100 μL Celltiter-Glo reagent was added to each well, and then the plate was shake for 2 minutes to fully lysate. Then the plate was incubated at room temperature for 10 minutes, and the chemiluminescence values were read by plate reader.

The inhibitory activity of compounds on cell proliferation was plotted using cell survival rate against the compound concentration as coordinates. Cell inhibition rate %=(Lum_{compound}−Lum_{DMSO})/(Lum_{medium}−Lum_{DMSO})×100. Lum refers to the chemiluminescence value.

XL Fit software was used to Fit nonlinear s-curve regression to Fit the data to obtain dose-effect curves, from which IC₅₀ values were calculated. Y=Bottom+(Top−Bottom)/(1+10\hat{ }((Log IC₅₀−X)×slope)), Where Y is the cell inhibition rate, X is the compound concentration, Bottom is the lowest inhibition rate. Top is the highest inhibition rate.

Table 2 summarizes the inhibitory effect data (IC₅₀) of the compounds on the proliferation of human breast cancer cells MDA-MB-436 determined by CTG method.

TABLE 2

| Example | IC₅₀ (nM) |
|---|---|
| 1 | 160.56 |
| 2 | >1000 |
| 3 | 57.61 |
| 14 | 21.65 |
| 22 | 226.53 |
| 23 | 496.15 |
| 27 | 157.50 |
| 28 | 40.61 |
| 40 | 21.09 |
| 41 | 55.31 |

CCK Method: After 7 days, 20 μL of CCK-8 was added to each well and shaken gently, then was cultured for 4 hours. The plate was shaken for 5 min after incubation, the absorbance values of 450 nm or 650 nm wavelengths were recorded respectively (OD=absorbance value of 450 nm−absorbance value of 650 nm) by using the multifunction readout instrument.

Data were analyzed by software GraphPad Prism 6.0. The inhibitory activity of compounds on cell proliferation was plotted using cell survival rate against the compound concentration as coordinates. Cell survival rate %=(OD_{compound}−OD_{background})/(OD_{DMSO}−OD_{background})×100. The IC₅₀ value was fitted by the s-shaped dose response curve equation: Y=100/(1+10\hat{ }(log C−log IC₅₀)), and C was the compound concentration.

Table 3 summarizes the inhibitory effect data (IC₅₀) of the compounds on the proliferation of human breast cancer cells MDA-MB-436 determined by CCK-8 method.

TABLE 3

| Example | IC₅₀ (nM) |
|---|---|
| 3 | 111.17 |
| 4 | 25.53 |
| 5 | 38.82 |
| 6 | 28.31 |
| 9 | 1172.2 |
| 10 | 225.94 |
| 14 | 26.36 |
| 15 | 10.26 |
| 16 | 13.24 |
| 17 | 39.45 |
| 19 | 44.77 |

Therefore, the compounds herein have a good inhibitory effect on the proliferation of human breast cancer cells MDA-MB-436.

Having now fully described this disclosure, it will be understood by those of ordinary skill in the art that the same can be performed within a wide and equivalent range of conditions, formulations and other parameters without affecting the scope of the disclosure or any embodiment thereof. All patents, patent applications and publications cited herein are fully incorporated by reference herein in their entirety.

1. A compound of Formula I: 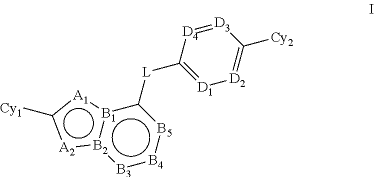
or stereoisomers, tautomers, N-oxides, hydrates, solvates, isotope-substituted derivatives, or pharmaceutically acceptable salts thereof, or mixtures thereof, or prodrugs thereof, wherein:
A₁ and A₂ are each independently selected from a group consisting of N and CR₁;
B₁ and B₂ are each independently selected from a group consisting of N and C, and at most one of B₁ and B₂ is N;
B₃, B₄ and B₅ are each independently selected from a group consisting of N and CR₂;
D₁, D₂, D₃ and D₄ are each independently selected from a group consisting of N and CR₃;
L is selected from a group consisting of NR₆, O, S, SO, SO₂, C═O and an alkylene optionally substituted with R₄ and/or R₅;
Cy₁ is selected from a group consisting of an optionally substituted carbocyclic group, an optionally substituted heterocyclic group, an optionally substituted aryl and an optionally substituted heteroaryl;
Cy₂ is selected from a group consisting of an optionally substituted carbocyclic group, an optionally substituted heterocyclic group, an optionally substituted aryl and an optionally substituted heteroaryl;
R₁, R₂ and R₃ are each independently selected from a group consisting of hydrogen, halogen, cyano, an optionally substituted alkyl, an optionally substituted alkoxy, an optionally substituted carbocyclic group, an optionally substituted alkenyl, an optionally substituted alkynyl, and an optionally substituted amino;
R₄ and R₅ are each independently selected from a group consisting of halogen, cyano, an optionally substituted alkyl, an optionally substituted alkoxy, an optionally substituted carbocyclic group, an optionally substituted alkenyl and an optionally substituted alkynyl; or R₄ and R₅ together with the attached C form a ring;
R₆ is selected from a group consisting of hydrogen and an optionally substituted alkyl.

2. The compound of Formula I as claimed in claim 1, or stereoisomers, tautomers, N-oxides, hydrates, solvates, isotope-substituted derivatives, or pharmaceutically acceptable salts thereof, or mixtures thereof, or prodrugs thereof, wherein:
A₁ is N and A₂ is N; and/or
B₁ is N, and B₂ is C; or B₁ is C, and B₂ is N; and/or
L is a C₁₋₃ alkylene group, NH, N—C₁₋₃ alkyl or O; and/or
R₆ is H or C₁₋₃ alkyl.

3. The compound of Formula I as claimed in claim 1, or stereoisomers, tautomers, N-oxides, hydrates, solvates, isotope-substituted derivatives, or pharmaceutically acceptable salts thereof, or mixtures thereof, or prodrugs thereof, wherein B₃, B₄ and B₅ are each independently selected from a group consisting of N and CR₂, wherein R₂ is H, halogen, C₁₋₄ alkyl or C₁₋₄ alkoxy.

4. The compound of Formula I as claimed in claim 1, or stereoisomers, tautomers, N-oxides, hydrates, solvates, isotope-substituted derivatives, or pharmaceutically acceptable salts thereof, or mixtures thereof, or prodrugs thereof, wherein:
D₁, D₂, D₃ and D₄ are each independently CR₃;
R₃ is selected from a group consisting of hydrogen, halogen, an optionally substituted alkyl and an optionally substituted alkoxy, wherein the said alkyl or the said alkoxy is optionally substituted by 1-5 substituents selected from the group consisting of halogen, hydroxyl and NRₐR_{b}, wherein the said Rₐ and R_{b} are independently H or C₁₋₄ alkyl.

5. The compound of Formula I as claimed in claim 1, or stereoisomers, tautomers, N-oxides, hydrates, solvates, isotope-substituted derivatives, or pharmaceutically acceptable salts thereof, or mixtures thereof, or prodrugs thereof, wherein;
Cy₁ is an optionally substituted C₃₋₈cycloalkyl, an optionally substituted 4-10 membered heterocyclic group, an optionally substituted 6-14 membered aryl group or an optionally substituted 5-10 membered heteroaryl group;
Cy₁ is optionally substituted by substituent(s) selected from the group consisting of halogen, an optionally substituted C₁₋₄ alkyl, an optionally substituted C₁₋₄ alkoxy, an optionally substituted C₃₋₆, cycloalkyl, an optionally substituted amino and cyano; and
each of the said C₁₋₄ alkyl, C₁₋₄ alkoxy and C₃₋₆ cycloalkyl are optionally substituted by 1-5 substituents selected from the group consisting of halogen, hydroxyl and C₁₋₄ alkyl substituted with —NRₐR_{b}, wherein the said Rₐ and R_{b} are independently H or C₁₋₄ alkyl; and the said amino is optionally substituted with 1 or 2 C₁₋₄ alkyl.

6. The compound of Formula I as claimed in claim 1, or stereoisomers, tautomers, N-oxides, hydrates, solvates, isotope-substituted derivatives, or pharmaceutically acceptable salts thereof, or mixtures thereof, or prodrugs thereof, wherein:
Cy₂ is an optionally substituted 6-14 membered aryl group, an optionally substituted 5-10 membered heteroaryl group, an optionally substituted C₃₋₈cycloalkyl or an optionally substituted 4-10 membered heterocyclic group;
Cy₂ is optionally substituted by 1-5 substituents selected from a group consisting of halogen, an optionally substituted C₁₋₄ alkyl, an optionally substituted C₁₋₄ alkoxy and an optionally substituted C₃₋₆ cycloalkyl; and
each of the said C₁₋₄ alkyl and C₁₋₄ alkoxy are optionally substituted by 1-5 substituents selected from the group consisting of halogen, hydroxyl and C₁₋₄ alkyl substituted with —NRₐR_{b}, wherein the said Rₐ and R_{b} are independently H or C₁₋₄ alkyl.

7. The compound of Formula I as claimed in claim 1, or stereoisomers, tautomers, N-oxides, hydrates, solvates, isotope-substituted derivatives, or pharmaceutically acceptable salts thereof, or mixtures thereof, or prodrugs thereof, wherein the compound of Formula I is a compound represented by Formula Ha or IIb: 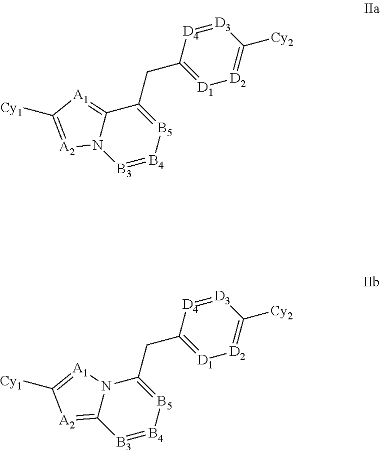
or stereoisomers, tautomers, N-oxides, hydrates, solvates, isotope-substituted derivatives, or pharmaceutically acceptable salts thereof, or mixtures thereof, or prodrugs thereof.

8. The compound as claimed in claim 1, or stereoisomers, tautomers, N-oxides, hydrates, solvates, isotope-substituted derivatives, or pharmaceutically acceptable salts thereof, or mixtures thereof, or prodrugs thereof, wherein the compound of Formula I is a compound represented b Formula IIIa or IIIb: 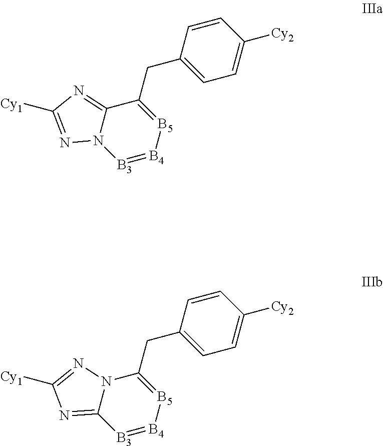
or stereoisomers, tautomers, N-oxides, hydrates, solvates, isotope-substituted derivatives, or pharmaceutically acceptable salts thereof, or mixtures thereof, or prodrugs thereof.

9. The compound of Formula I as claimed in claim 1, wherein the compound is selected from a group consisting of:
8-(4-(1,4-dimethyl-1H-imidazol-2-yl)benzyl)-2-(2-isopropylphenyl)-[1,2,4]triazolo[1,5-a]pyridine;
8-(4-(1,4-dimethyl-1H-imidazol-2-yl)benzyl)-2-(2-methoxyphenyl)-[1,2,4]triazolo[1,5-a]pyridine;
2-(2-isopropylphenyl)-8-(4-(1-methyl-4-(trifluoromethyl)-1H-imidazol-2-yl)benzyl)-[1,2,4]triazolo[1,5-a]pyridine;
2-(4-cyclopropyl-6-methoxypyrimidin-5-yl)-8-(4-(1-isopropyl-4-(trifluoromethyl)-1H-imidazol-2-yl)benzyl)-[1,2,4]triazolo[1,5-a]pyridine;
2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-a]pyridine;
2-(4-cyclopropyl-6-methoxypyrimidin-5-yl)-8-(4-(5-methyl-3-(trifluoromethyl)-1H-pyrazol-1-yl)benzyl)-[1,2,4]triazolo[1,5-a]pyridine;
2-(2-isopropylphenyl)-8-(4-(1-methyl-4-(trifluoromethyl)-1H-imidazol-2-yl)benzyl)-[1,2,4]triazolo[1,5-a]pyrazine;
2-(2-isopropylphenyl)-8-(4-(1-methyl-4-(trifluoromethyl)-1H-imidazol-2-yl)benzyl)-[1,2,4]triazolo[1,5-c]pyrimidine;
2-(2-isopropylphenyl)-8-(4-(1-methyl-4-(trifluoromethyl)-1H-imidazol-2-yl)benzyl)-[1,2,4]triazolo[1,5-b]pyridazine;
2-(2-isopropylphenyl)-5-(4-(1-methyl-4-(trifluoromethyl)-1H-imidazol-2-yl)benzyl)-[1,2,4]triazolo[1,5-a]pyridine;
2-(2-isopropylphenyl)-7-(4-(1-methyl-4-(trifluoromethyl)-1H-imidazol-2-yl)benzyl)-[1,2,4]triazolo[1,5-a]pyrimidine;
2-(2-isopropylphenyl)-5-(4-(1-methyl-4-(trifluoromethyl)-1H-imidazol-2-yl)benzyl)-[1,2,4]triazolo[1,5-a]pyrazine;
2-(2-isopropylphenyl)-5-(4-(1-methyl-4-(trifluoromethyl)-1H-imidazol-2-yl)benzyl)-[1,2,4]triazolo[1,5-c]pyrimidine;
2-(4-cyclopropyl-6-methoxypyrimidin-5-yl)-5-(4-(1-methyl-4-(trifluoromethyl)-1H-imidazol-2-yl)benzyl)-[1,2,4]triazolo[1,5-a]pyridine;
2-(1-isopropyl-4-methyl-1H-pyrazol-5-yl)-5-(4-(1-methyl-4-(trifluoromethyl)-1H-imidazol-2-yl)benzyl)-[1,2,4]triazolo[1,5-a]pyridine;
2-(1-isopropyl-4-methyl-1H-pyrazol-5-yl)-8-(4-(1-methyl-4-(trifluoromethyl)-1H-imidazol-2-yl)benzyl)-[1,2,4]triazolo[1,5-a]pyridine;
2-(1-isopropyl-4-methyl-1H-pyrazol-5-yl)-8-(4-(1-methyl-4-(trifluoromethyl)-1H-imidazol-2-yl)benzyl)-[1,2,4]triazolo[1,5-b]pyridazine;
2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-a]pyrazine;
2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-c]pyrimidine;
2-(4-cyclopropyl-6-methoxypyrimidin-5-yl)-8-(4-(1-methyl-4-(trifluoromethyl)-1H-imidazol-2-yl)benzyl)-[1,2,4]triazolo[1,5-b]pyridazine;
2-(4-cyclopropyl-6-methoxypyrimidin-5-yl)-5-(4-(1-methyl-4-(trifluoromethyl)-1H-imidazol-2-yl)benzyl)-[1,2,4]triazolo[1,5-c]pyrimidine;
2-(4-cyclopropyl-6-methoxypyrimidin-5-yl)-5-(4-(1-methyl-4-(trifluoromethyl)-1H-imidazol-2-yl)benzyl)-[1,2,4]triazolo[1,5-a]pyrazine;
2-(4-cyclopropyl-6-methoxypyrimidin-5-yl)-7-(4-(1-methyl-4-(trifluoromethyl)-1H-imidazol-2-yl)benzyl)-[1,2,4]triazolo[1,5-a]pyrimidine;
8-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-a]pyridine;
2-(4-cyclopropyl-6-methoxypyrimidin-5-yl)-8-(3-fluoro-4-(1-methyl-4-(trifluoromethyl)-1H-imidazol-2-yl)benzyl)-[1,2,4]triazolo[1,5-a]pyridine;
2-(4-cyclopropyl-6-methoxypyrimidin-5-yl)-8-(4-(1-ethyl-4-(trifluoromethyl)-1H-imidazol-2-yl)-3-fluoro-5-methoxybenzyl)-[1,2,4]triazolo[1,5-a]pyridine;
2-(4,6-dimethoxypyrimidin-5-yl)-8-(4-(1-methyl-4-(trifluoromethyl)-1H-imidazol-2-yl)benzyl)-[1,2,4]triazolo[1,5-a]pyridine;
2-(4-cyclobutyl-6-methoxypyrimidin-5-yl)-8-(4-(1-methyl-4-(trifluoromethyl)-1H-imidazol-2-yl)benzyl)-[1,2,4]triazolo[1,5-a]pyridine;
8-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-a]pyrazine;
2-(4-cyclopropyl-6-methoxypyrimidin-5-yl)-8-(3-fluoro-4-(1-methyl-4-(trifluoromethyl)-1H-imidazol-2-yl)benzyl)-[1,2,4]triazolo[1,5-a]pyrazine;
2-(4-cyclopropyl-6-methoxypyrimidin-5-yl)-8-(4-(1-ethyl-4-(trifluoromethyl)-1H-imidazol-2-yl)-3,5-difluorobenzyl)-[1,2,4]triazolo[1,5-a]pyrazine;
8-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-c]pyrimidine;
2-(4-cyclopropyl-6-methoxypyrimidin-5-yl)-8-(3-fluoro-4-(1-methyl-4-(trifluoromethyl)-1H-imidazol-2-yl)benzyl)-[1,2,4]triazolo[1,5-c]pyrimidine;
2-(4-cyclopropyl-6-methoxypyrimidin-5-yl)-8-(4-(1-ethyl-4-(trifluoromethyl)-1H-imidazol-2-yl)-3,5-difluorobenzyl)-[1,2,4]triazolo[1,5-c]pyrimidine;
2-(4-cyclopropyl-6-cyanopyrimidin-5-yl)-8-(4-(1-methyl-4-(trifluoromethyl)-1H-imidazol-2-yl)benzyl)-[1,2,4]triazolo[1,5-a]pyridine;
2-(4-cyclopropyl-6-methoxypyrimidin-5-yl)-8-(1-(4-(1-methyl-4-(trifluoromethyl)-1H-imidazol-2-yl)phenyl)ethyl)-[1,2,4]triazolo[1,5-a]pyridine;
5-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-a]pyridine;
2-(4-cyclopropyl-6-methoxypyrimidin-5-yl)-5-(3-fluoro-4-(1-methyl-4-(trifluoromethyl)-1H-imidazol-2-yl)benzyl)-[1,2,4]triazolo[1,5-a]pyridine;
2-(4-cyclopropyl-6-methoxypyrimidin-5-yl)-5-(4-(1-ethyl-4-(trifluoromethyl)-1H-imidazol-2-yl)-3-fluoro-5-methoxybenzyl)-[1,2,4]triazolo[1,5-a]pyridine;
5-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-c]pyrimidine;
2-(4-cyclopropyl-6-methoxypyrimidin-5-yl)-5-(3-fluoro-4-(1-methyl-4-(trifluoromethyl)-1H-imidazol-2-yl)benzyl)-[1,2,4]triazolo[1,5-c]pyrimidine;
2-(4-cyclopropyl-6-methoxypyrimidin-5-yl)-5-(4-(1-ethyl-4-(trifluoromethyl)-1H-imidazol-2-yl)-3,5-difluorobenzyl)-[1,2,4]triazolo[1,5-c]pyrimidine;
5-(4-(1-cyclopropyl-4-(trifluoromethyl)-1H-imidazol-2-yl)benzyl)-2-(4-cyclopropyl-6-methoxypyrimidin-5-yl)-[1,2,4]triazolo[1,5-a]pyrazine;
2-(4-cyclopropyl-6-methoxypyrimidin-5-yl)-5-(3-fluoro-4-(1-methyl-4-(trifluoromethyl)-1H-imidazol-2-yl)benzyl)-[1,2,4]triazolo[1,5-a]pyrazine;
2-(4-cyclopropyl-6-methoxypyrimidin-5-yl)-5-(4-(1-ethyl-4-(trifluoromethyl)-1H-imidazol-2-yl)-3,5-difluorobenzyl)-[1,2,4]triazolo[1,5-a]pyrazine;
2-(4-cyclopropyl-6-cyanopyrimidin-5-yl)-5-(4-(1-methyl-4-(trifluoromethyl)-1H-imidazol-2-yl)benzyl)-[1,2,4]triazolo[1,5-a]pyridine;
2-(4-cyclopropyl-6-methoxypyrimidin-5-yl)-5-(1-(4-(1-methyl-4-(trifluoromethyl)-1H-imidazol-2-yl)phenyl)ethyl)-[1,2,4]triazolo[1,5-a]pyridine;
or stereoisomers, tautomers, N-oxides, hydrates, isotope-substituted derivatives, solvates or pharmaceutically acceptable salts thereof, or mixtures thereof, or prodrugs thereof.

10.-13. (canceled)

14. A pharmaceutical composition comprising the compound of Formula I as claimed in claim 1, or stereoisomers, tautomers, N-oxides, hydrates, isotope-substituted derivatives, solvates or pharmaceutically acceptable salts thereof, or mixtures thereof, or prodrugs thereof, and a pharmaceutically acceptable carrier.

15. The pharmaceutical composition of claim 14, wherein the composition further includes at least one known anticancer drug or pharmaceutically acceptable salts thereof, selected from a group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, Ofatumumab, Dinutuximab, Blinatumomab, ipilimumab, avastin, herceptin, mabthera, T-DM1, Trastuzumab Deruxtecan, Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, Belantamab Mafodotin, imatinib, gefitinib, erlotinib, osimertinib, afatinib, ceritinib, alectinib, crizotinib, erlotinib, lapatinib, sorafenib, sunitinib, nilotinib, dasatinib, pazopanib, torisel, everolimus, vorinostat, romidepsin, panobinostat, belinostat, tamoxifen, letrozole, fulvestrant, mitoguazone, octreotide, retinoic acid, arsenic trioxide, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine), palbociclib, olaparib, niraparib, rucaparib, talazoparib and senaparib.

16. The compound of Formula I as claimed in claim 3, or stereoisomers, tautomers, N-oxides, hydrates, solvates, isotope-substituted derivatives, or pharmaceutically acceptable salts thereof, or mixtures thereof, or prodrugs thereof, wherein:
B₃, B₄ and B₅ are each independently N or CH; or all of B₃, B₄ and B₅ are CR₂, wherein R₂ is each independently H, halogen or C₁₋₄ alkyl; or
B₃ is N, both B₄ and B₅ are CR₂, wherein R₂ is each independently H, halogen or C₁₋₄ alkyl; or
B₄ is N, both B₃ and B₅ are CR₂, wherein R₂ is each independently H, halogen or C₁₋₄ alkyl; or B₅ is N, both B₃ and B₄ are CR₂, wherein R₂ is each independently H, halogen or C₁₋₄ alkyl; or
the fused heteroaromatic bicyclic ring containing A₁, A₂, B₁, B₂, B₃, B₄ and B₅ is selected from a group consisting of: 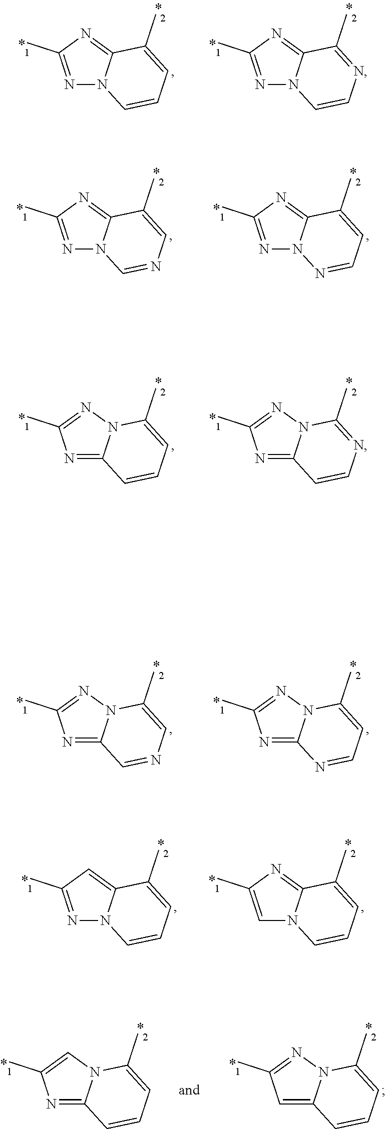
wherein, *1 and *2 refer to the position of attachment of the group to Cy₁ and L of the compound, respectively;

17. The compound of Formula I as claimed in claim 4, or stereoisomers, tautomers, N-oxides, hydrates, solvates, isotope-substituted derivatives, or pharmaceutically acceptable salts thereof, or mixtures thereof, or prodrugs thereof, wherein
D₁ and D₄ are CH, D₂ and D₃ are CR₃, wherein each R₃ is independently hydrogen, halogen or C₁₋₄ alkoxy; or
D₁ and D₄ are CH, D₂ and D₃ are CR₃, wherein each R₃ is independently hydrogen, halogen or C₁₋₄ alkoxy, and at least one of R₃ is a non-hydrogen substituent.

18. The compound of Formula I as claimed in claim 5, or stereoisomers, tautomers, N-oxides, hydrates, solvates, isotope-substituted derivatives, or pharmaceutically acceptable salts thereof, or mixtures thereof, or prodrugs thereof, wherein:
Cy₁ is an optionally substituted phenyl, an optionally substituted pyridyl, an optionally substituted pyrimidinyl, an optionally substituted pyrazinyl, an optionally substituted pyridazinyl, an optionally substituted piperidinyl, an optionally substituted piperazinyl, an optionally substituted tetrahydrofuranyl, an optionally substituted pyrrolidinyl or an optionally substituted pyrazolyl.

19. The compound of Formula I as claimed in claim 6, or stereoisomers, tautomers, N-oxides, hydrates, solvates, isotope-substituted derivatives, or pharmaceutically acceptable salts thereof, or mixtures thereof, or prodrugs thereof, wherein Cy₂ is imidazolyl substituted with an optionally substituted C₁₋₄ alkyl or pyrazolyl substituted with an optionally substituted C₁₋₄ alkyl.

20. A method for treating or preventing an USP1 regulation related disease, comprising administering to an object in need an effective amount of the compound of Formula I of claim 1 or stereoisomers, tautomers, N-oxides, hydrates, solvates, isotope-substituted derivatives, or pharmaceutically acceptable salts thereof, or mixtures thereof, or prodrugs thereof.

21. The method as claimed in claim 20, wherein the disease is cancer.

22. The method as claimed in claim 21, wherein the cancer is liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, Wilms tumor, cervical cancer, testicular cancer, soft tissue sarcoma, primary macroglobulinemia, bladder cancer, chronic myeloid leukemia, primary brain cancer, malignant melanoma, non-small lung cancer, small cell lung cancer, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, or prostate cancer.

23. The method as claimed in claim 21, wherein the method further comprises radiotherapy.

24. The method as claimed in claim 21, wherein the method further comprises administering at least one known anticancer drug or a pharmaceutically acceptable salt thereof selected from a group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, Ofatumumab, Dinutuximab, Blinatumomab, ipilimumab, avastin, herceptin, mabthera, T-DM1, Trastuzumab Deruxtecan, Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, Belantamab Mafodotin, imatinib, gefitinib, erlotinib, osimertinib, afatinib, ceritinib, alectinib, crizotinib, erlotinib, lapatinib, sorafenib, sunitinib, nilotinib, dasatinib, pazopanib, torisel, everolimus, vorinostat, romidepsin, panobinostat, belinostat, tamoxifen, letrozole, fulvestrant, mitoguazone, octreotide, retinoic acid, arsenic trioxide, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine), palbociclib, olaparib, niraparib, rucaparib, talazoparib and senaparib.
